(19) United States
(12) Patent Application Publication
(10) Pub. No.: US 2010/0164371 A1
(43) Pub. Date: Jul. 1, 2010

(54) ELECTRON TRANSPORTING-INJECTION COMPOUND AND ORGANIC ELECTROLUMINESCENT DEVICE USING THE SAME

(76) Inventors: Hyun Cheol JEONG, Gamdang-ri (KR); Dong Hee YOO, Seoul (KR); Jong Hyun PARK, Seoul (KR); Tae Han PARK, Seoul (KR); Kyung Hoon LEE, Goyang-si (KR)

Correspondence Address: BRINKS HOFER GILSON & LIONE, P.O. BOX 10395, CHICAGO, IL, 60610, US

(21) Appl. No.: 12/638,585

(22) Filed: Dec. 15, 2009

(30) Foreign Application Priority Data
Dec. 16, 2008 (KR) 10-2008-0128026

(51) Int. Cl. H01J 1/63; C07D 213/72; C07D 215/38

(52) U.S. Cl. 313/504; 546/304; 546/160

(57) ABSTRACT

An electron transporting-injection compound, represented by following Formula 1:

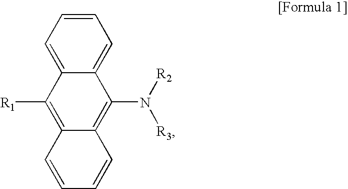

wherein each of the R1, the R2 and the R3 is selected from substituted or non-substituted aromatic group, substituted or non-substituted heterocyclic group, or of substituted or non-substituted aliphatic group, and at least one of the R2 and the R3 is selected from substituted or non-substituted heterocyclic group.

The present application claims the benefit of Korean Patent Application No. 10-2008-0128026 filed in Korea on Dec. 16, 2008, which is hereby incorporated by reference in its entirety.

### BACKGROUND

1. Field of the Disclosure

The present disclosure relates to an electron transporting-injection compound and an organic electroluminescent device (OELD) and more particularly to an electron transporting-injection compound having high luminescent efficiency and an OELD using the red phosphorescent compound.

2. Discussion of the Related Art

Recently, the requirement for a flat panel display device having a relatively large display area and a relatively small occupancy has been increased. Among the flat panel display devices, an OELD has various advantages as compared to an inorganic electroluminescent device, a liquid crystal display device, a plasma display panel, and so on. The OELD device has excellent characteristics of a view angel, a contrast ratio and so on. Also, since the OELD device does not require a backlight assembly, the OELD device has low weight and low power consumption. Moreover, the OELD device has advantages of a high response rate, a low production cost and so on.

In general, the OELD emits light by injecting electrons from a cathode and holes from an anode into an emission compound layer, combining the electrons with the holes, generating an exciton, and transforming the exciton from an excited state to a ground state. A flexible substrate, for example, a plastic substrate, can be used as a base substrate where elements are formed. The OELD has excellent characteristics of a view angel, a contrast ratio and so on. Also, since the OELD does not require a backlight assembly, the OELD has low weight and low power consumption. Moreover, the OELD has advantages of a high response rate, a low production cost, high color purity, etc. The OELD can be operated at a voltage (e.g., 10V or below) lower than a voltage required to operate other display devices. In addition, the OELD is adequate to produce full-color images.

A general method for fabricating OELDs will be briefly explained below. First, an anode is formed on a substrate by depositing a transparent conductive compound, for example, indium-tin-oxide (ITO). Next, a hole injection layer (HIL) is formed on the anode. For example, the HIL may be formed of copper phthalocyanine (CuPC), and have a thickness of about 10 nm to about 30 nm. Next, a hole transporting layer (HTL) is formed on the HIL. For example, the HTL may be formed of 4,4′-bis[N-(1-naphtyl)-N-phenylamino]-biphenyl (NPB or NPD) and have a thickness of about 30 nm to about 60 nm. Next, an emitting compound layer (EML) is formed on the HTL. A dopant may be doped onto the EML.

Next, an electron transporting layer (ETL) and an electron injection layer (EIL) are stacked on the EML. For example, the ETL may be formed of tris(8-hydroxy-quinolate)aluminum (Alq3). A cathode is formed on the EIL, and a passivation layer is formed on the cathode.

As mentioned above, the organic electroluminescent diode includes the anode, the HIL, the HTL, the EML, the ETL, the EIL, and the cathode, and Alq3 is used for the ETL. Unfortunately, Alq3 having a metal complex structure requires a relatively high driving voltage and produces a relatively low efficiency. Accordingly, there is requirement for development of an electron transporting compound having high efficiency and brightness.

To obtain a high current efficiency, a high internal quantum efficiency is required. Particularly, as shown in FIG. 1, as the blue color purity of an OELD becomes higher (i.e. as the Y index on CIE chromaticity coordinates decreases), the relative spectral sensitivity of images from the OELD decreases. Accordingly, it is difficult to achieve high luminescent efficiency of the OELD.

### SUMMARY

An electron transporting-injection compound is represented by following Formula 1:

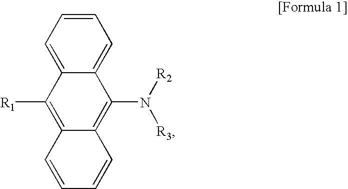

wherein each of the R1, the R2 and the R3 is selected from substituted or non-substituted aromatic group, substituted or non-substituted heterocyclic group, or of substituted or non-substituted aliphatic group, and at least one of the R2 and the R3 is selected from substituted or non-substituted heterocyclic group.

In another aspect, an electron transporting-injection compound is represented by following Formula 1:

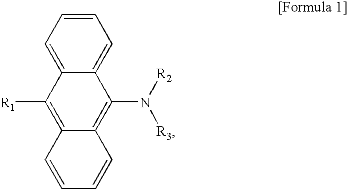

wherein each of the R1, the R2 and the R3 is selected from substituted or non-substituted aromatic group, substituted or non-substituted heterocyclic group, or of substituted or non-substituted aliphatic group, and at least one of the R2 and the R3 is selected from substituted or non-substituted heterocyclic group.

In another aspect, an organic electroluminescent device including a first electrode; a second electrode facing the first electrode; and an organic emitting layer positioned between the first and second electrodes and including a hole injection layer on the first electrode, a hole transporting layer on the hole injection layer, an emitting material layer on the hole injection layer and an electron transporting-injection layer on the emitting material layer, wherein the electron transporting-injection layer formed of an electron transporting-injection compound represented by following Formula 1:

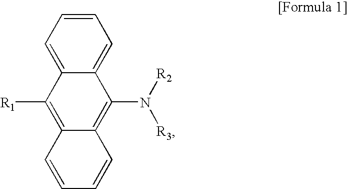

wherein each of the R1, the R2 and the R3 is selected from substituted or non-substituted aromatic group, substituted or non-substituted heterocyclic group, or of substituted or non-substituted aliphatic group, and at least one of the R2 and the R3 is selected from substituted or non-substituted heterocyclic group.

In another aspect, an organic electroluminescent device includes a first electrode; a second electrode facing the first electrode; and an organic emitting layer positioned between the first and second electrodes and including a hole injection layer on the first electrode, a hole transporting layer on the hole injection layer, an emitting material layer on the hole injection layer and an electron transporting-injection layer on the emitting material layer, wherein the electron transporting-injection layer formed of an electron transporting-injection compound represented by following Formula 1:

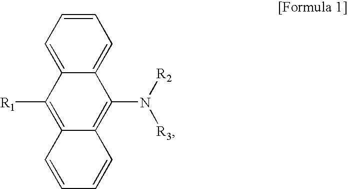

wherein each of the R1, the R2 and the R3 is selected from substituted or non-substituted aromatic group, substituted or non-substituted heterocyclic group, or of substituted or non-substituted aliphatic group, and at least one of the R2 and the R3 is selected from substituted or non-substituted heterocyclic group.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1 is a graph showing a relation of a color purity and a visible degree; and

FIG. 2 is a schematic cross-sectional view of an OELD according to the present invention.

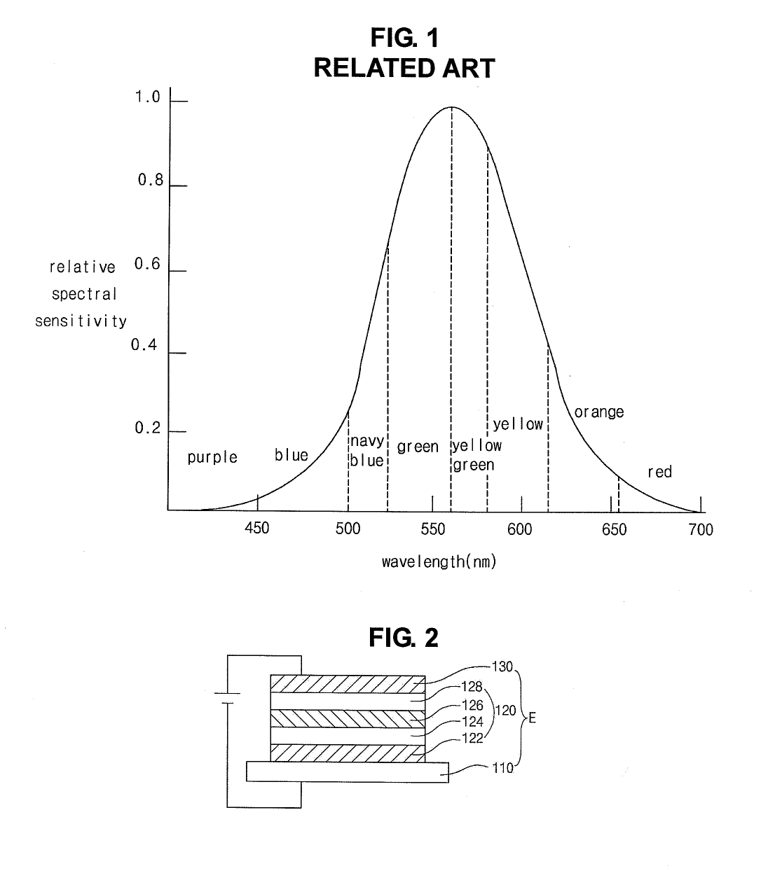

### DETAILED DESCRIPTION OF THE PREFERRED EMBODIMENTS

Reference will now be made in detail to the preferred embodiments, examples of which are illustrated in the accompanying drawings.

### First Embodiment

An electron transporting-injection compound according to the first embodiment of the present disclosure includes an asymmetric anthracene structure. In more detail, one side position of the anthracene is substituted by an ammonium salt, which is substituted by one of substituted or non-substituted aromatic group, substituted or non-substituted heterocyclic group, or of substituted or non-substituted aliphatic group, and the other side position of the anthracene is substituted by one of substituted or non-substituted aromatic group, substituted or non-substituted heterocyclic group, or of substituted or non-substituted aliphatic group. As a result, an organic electroluminescent diode including the electron transporting-injection compound according to the first embodiment of the present invention can have high luminescent efficiency, low driving voltage and long lifetime.

The electron transporting-injection compound according to the first embodiment of the present disclosure is represented by following Formula 1.

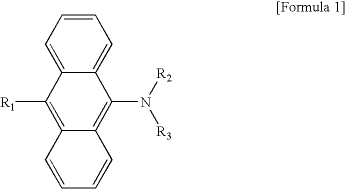

In the above Formula 1, each of R1, R2, and R3 is selected from substituted or non-substituted aromatic group, substituted or non-substituted heterocyclic group, or of substituted or non-substituted aliphatic group, and at least one of R2 and R3 is selected from substituted or non-substituted heterocyclic group.

In addition, the substituted or non-substituted heterocyclic group for at least one of R2 and R3 is pyridyl, and the electron transporting-injection compound according to the first embodiment has a following structure.

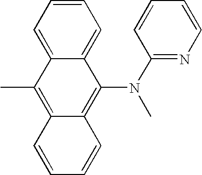

Since the compound is substituted by pyridyl to have a structure of

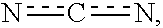

an electron attraction strength is increased such that the electron transporting-injection compound according to the present invention has improved properties for transporting and injecting an electron. As a result, luminescent efficiency is improved. The electron transporting-injection compound has an amorphous property due to an asymmetric structure such that a property of film is improved.

For example, the aromatic group includes phenyl, byphenyl, naphthyl, phenanthrenyl, and terphenyl, and the heterocyclic group includes pyridyl, bipyridyl, phenylpyridyl, pyridylphenyl, terpyridyl, quinolinyl, isoquinolinyl, and quinoxalinyl. The aliphatic group includes methyl, ethyl, propyl, isopropyl, butyl, and tert-butyl.

A substituent for each of R1, R2 and R3 is one of aryl, alkyl, alkoxy, allyamino, alkylamino, amino, halogen and cyano. For example, the substituent for each of R1, R2 and R3 is one of methyl, ethyl, propyl, isopropyl, tert-butyl, methoxy, ethoxy, butoxy, trimethylsilyl, fluorine and chlorine.

When at least one of R1, R2 and R3 is substituted by naphthyl, such as

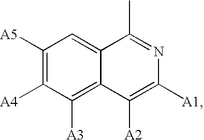

at least one of A1 to A5 is methyl. Alternatively, when at least one of R1, R2 and R3 is substituted by naphthyl, such as

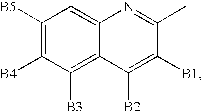

at least one of B1 to B5 is methyl. When the electron transporting-injection compound includes naphthyl substituted at least one methyl, luminescent efficiency and lifetime is further improved.

For example, the electron transporting-injection compound represented by Formula 1 is one of compounds in following Formula 2. For convenience, A-01 to A-216 are respectively marked to compounds.

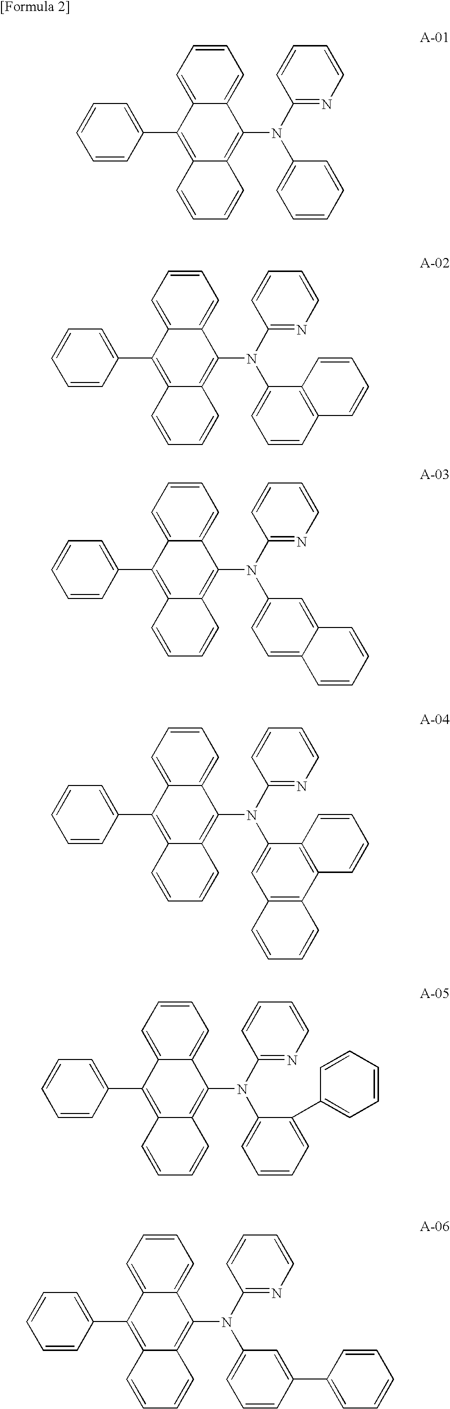

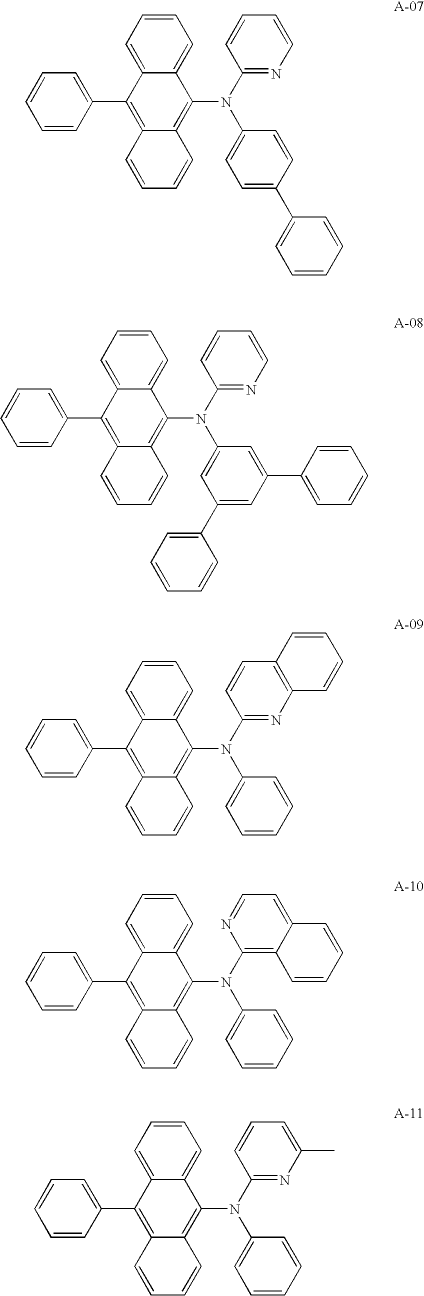

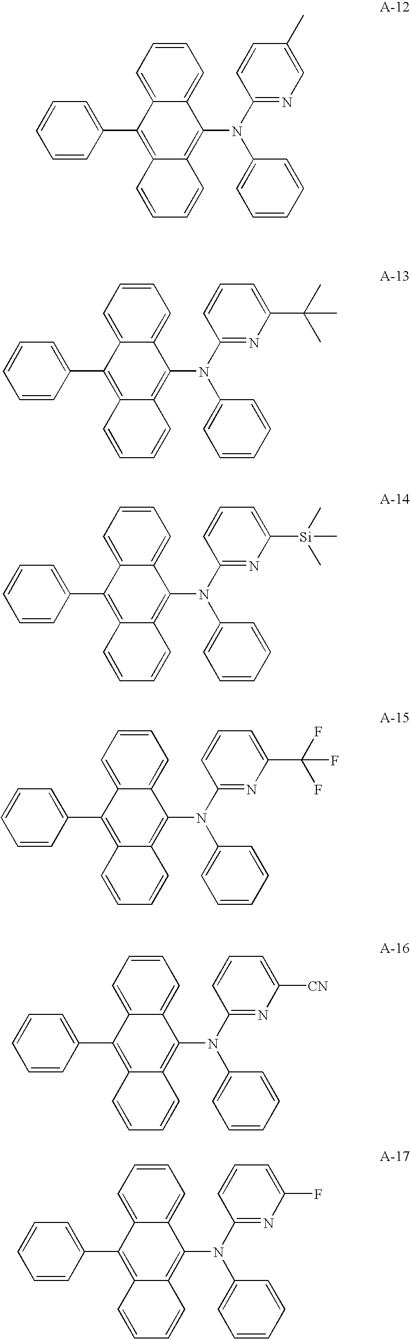

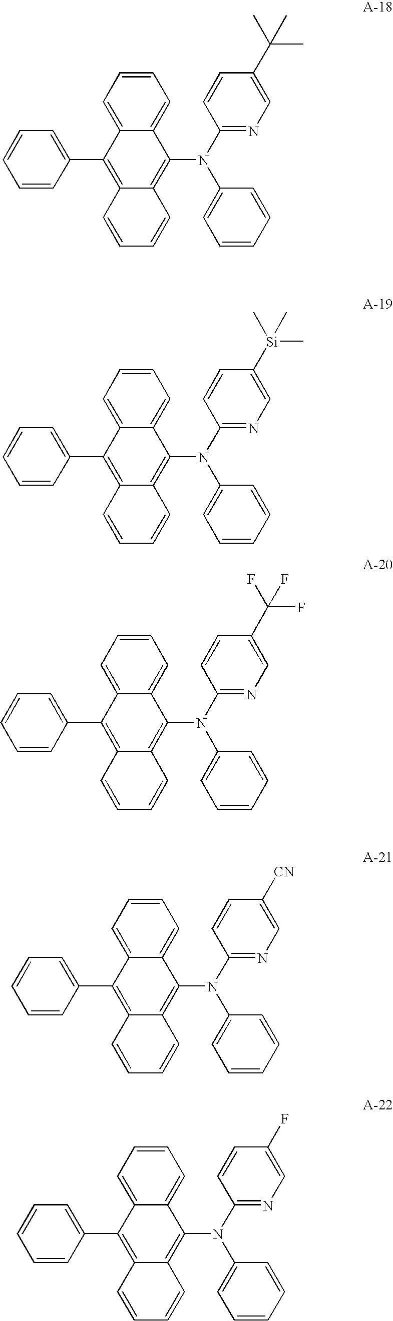

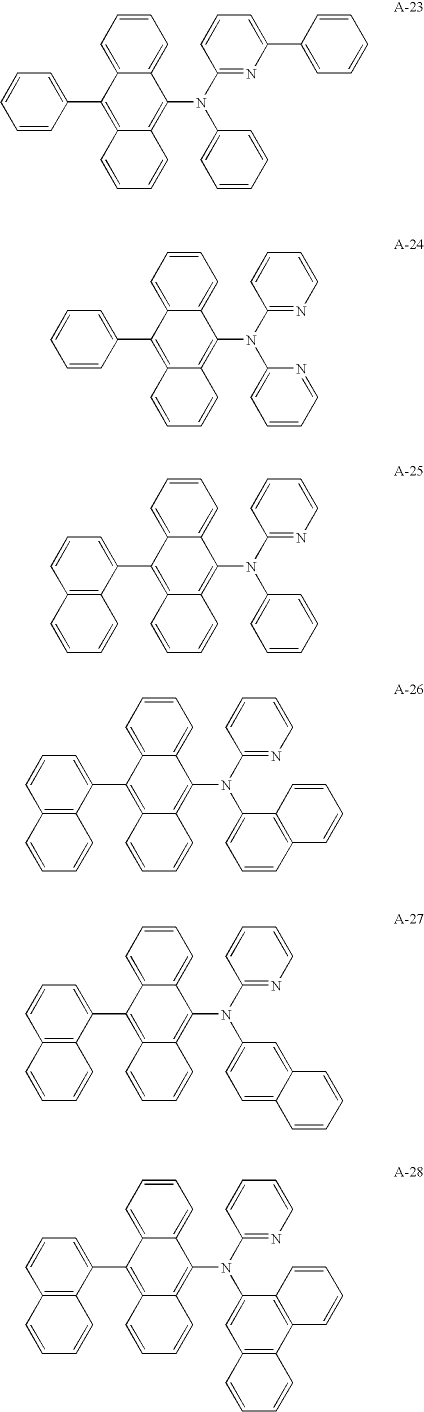

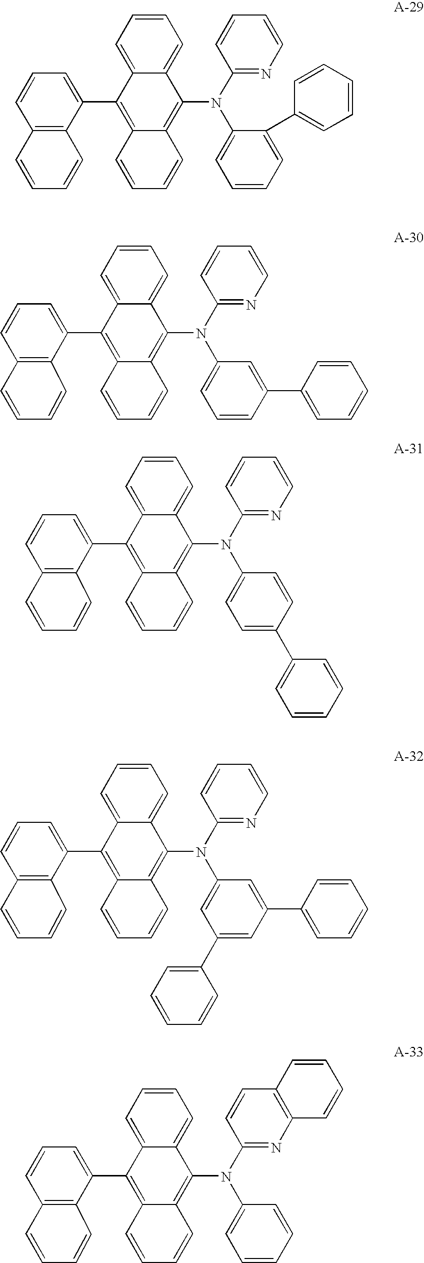

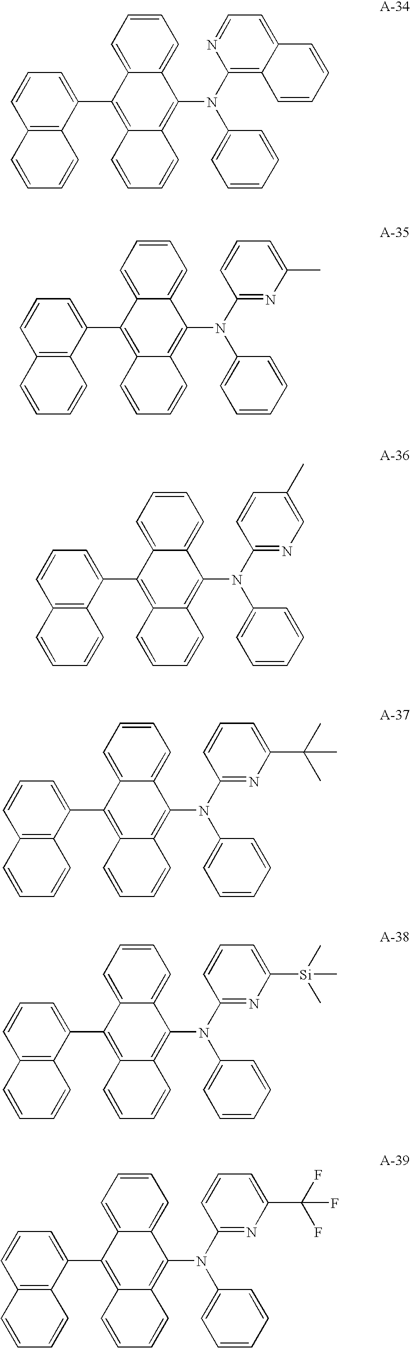

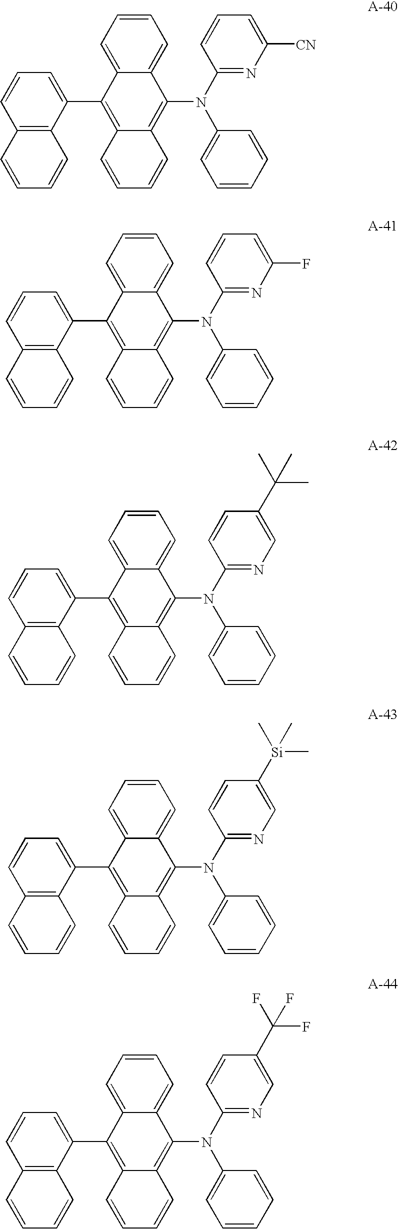

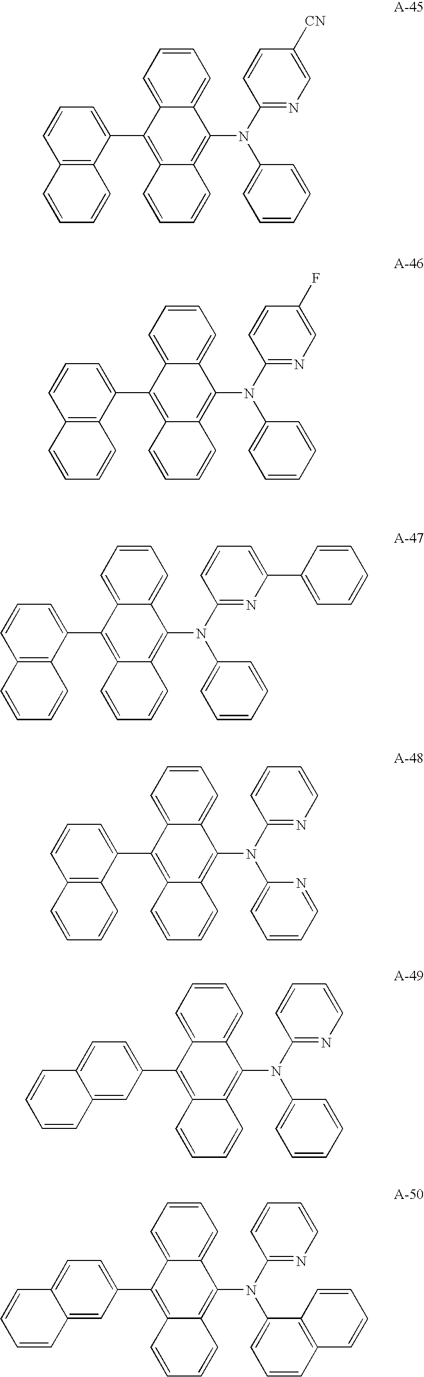

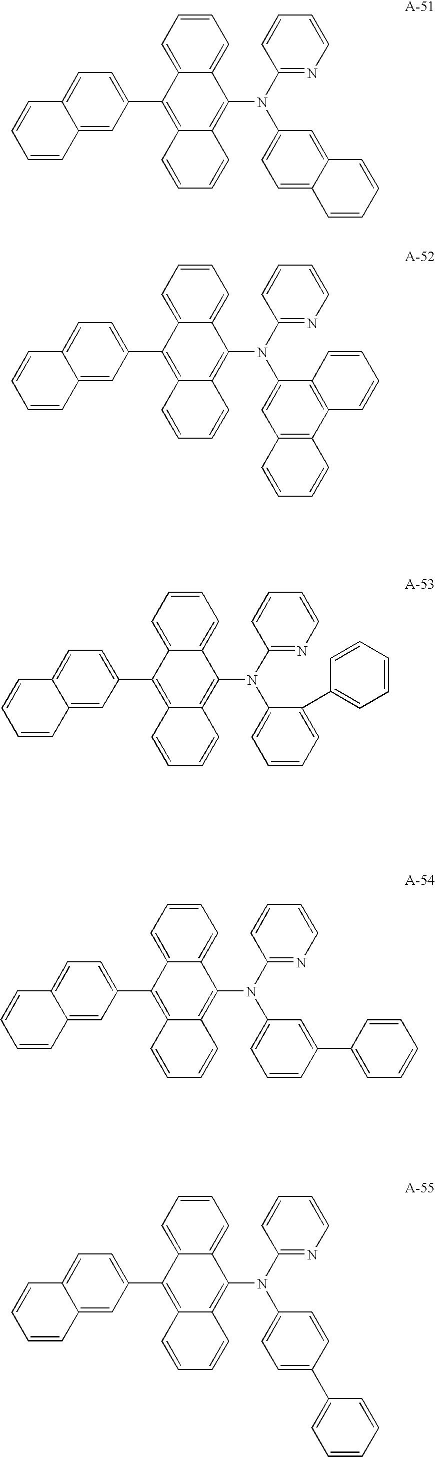

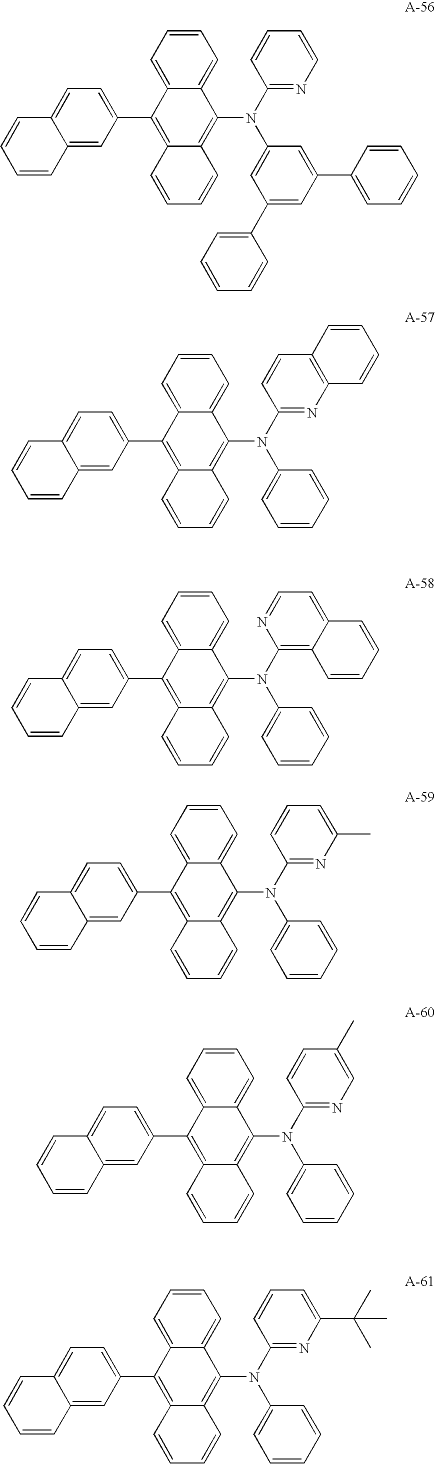

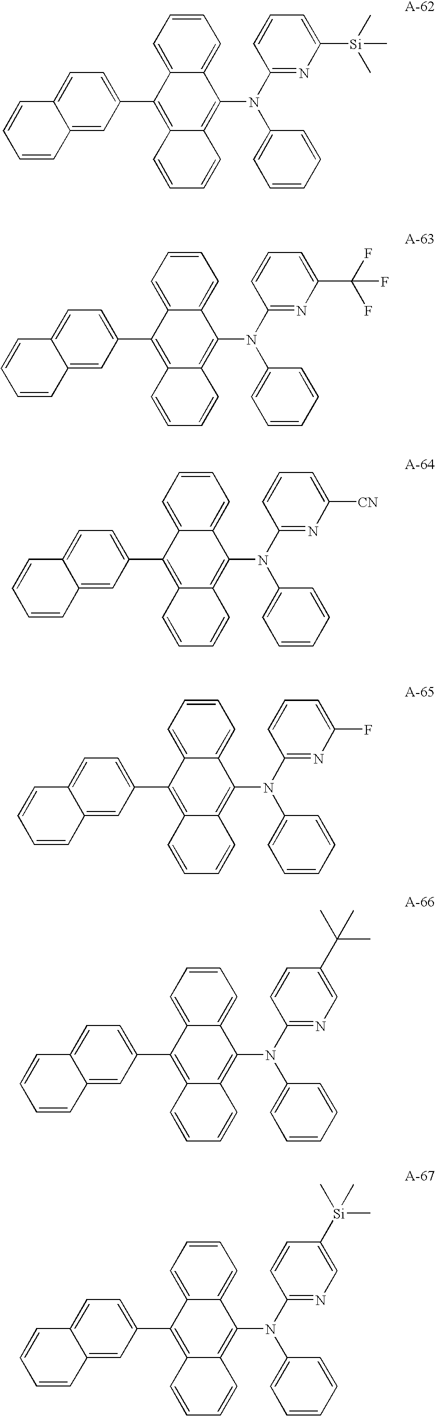

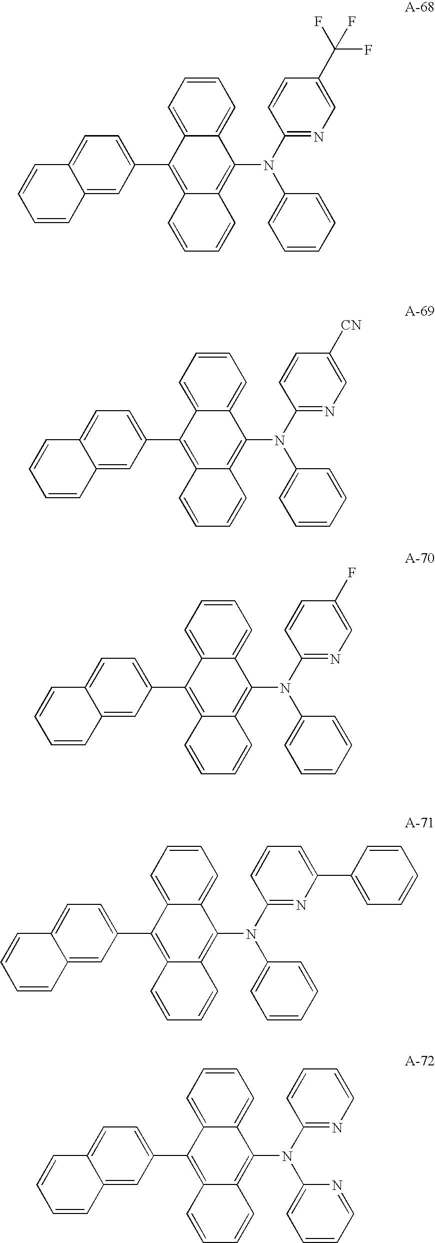

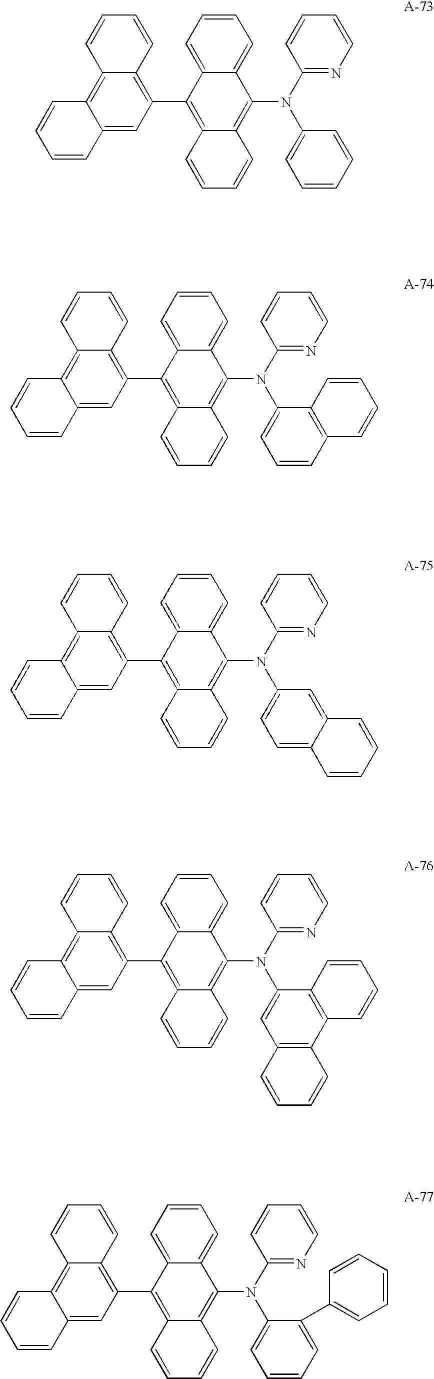

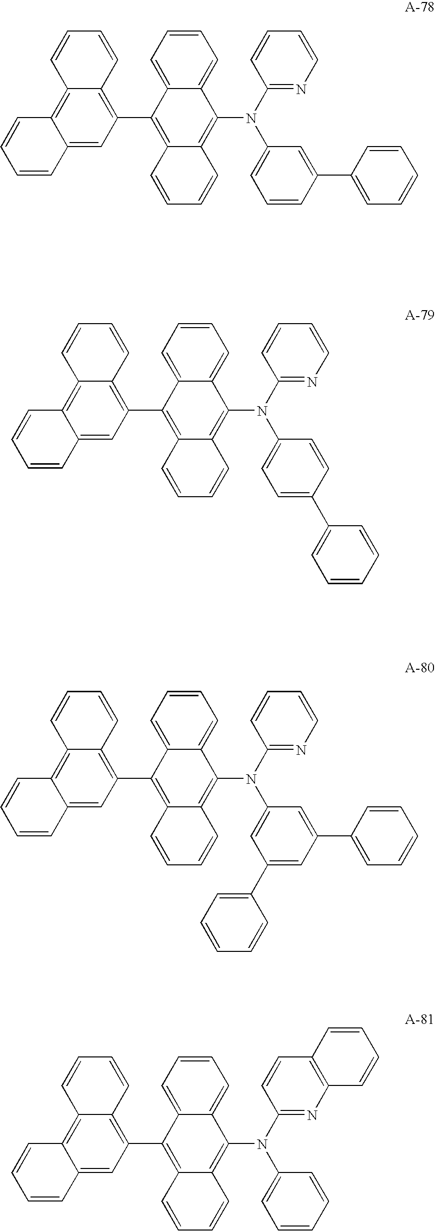

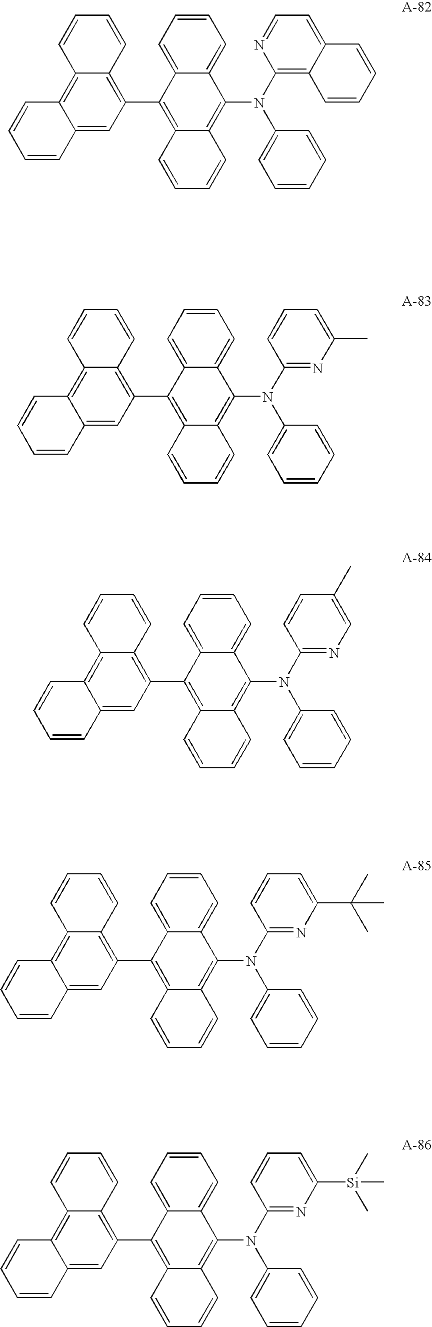

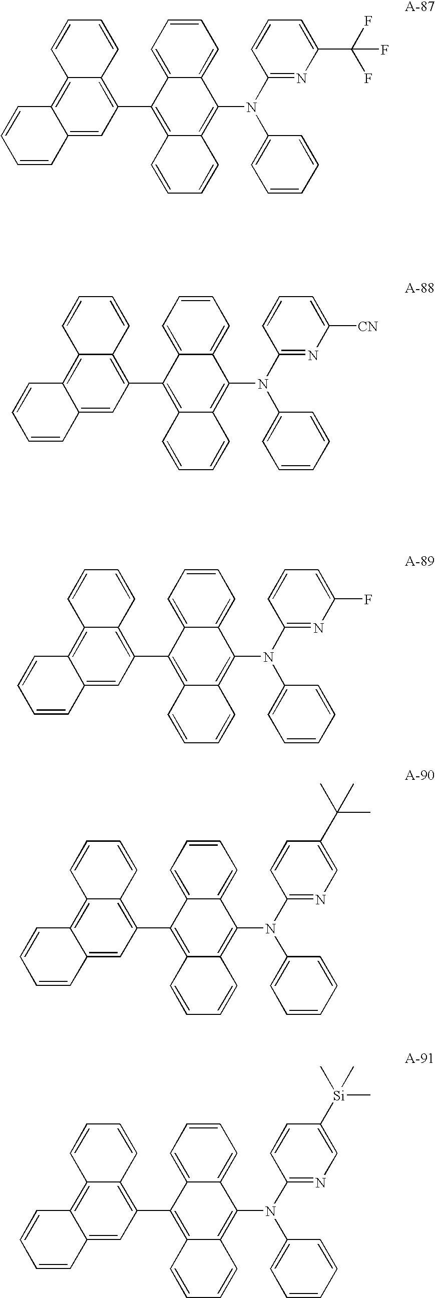

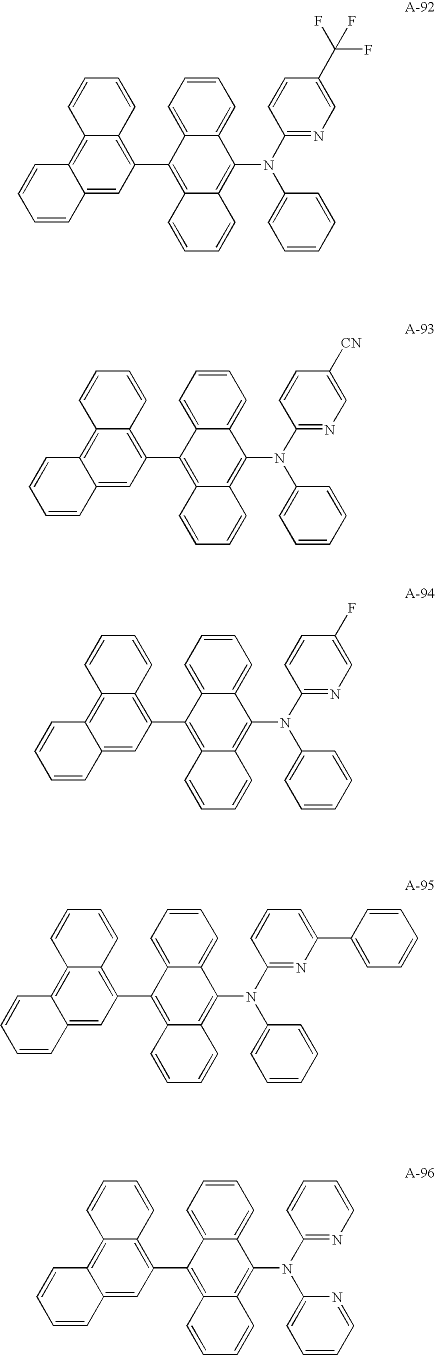

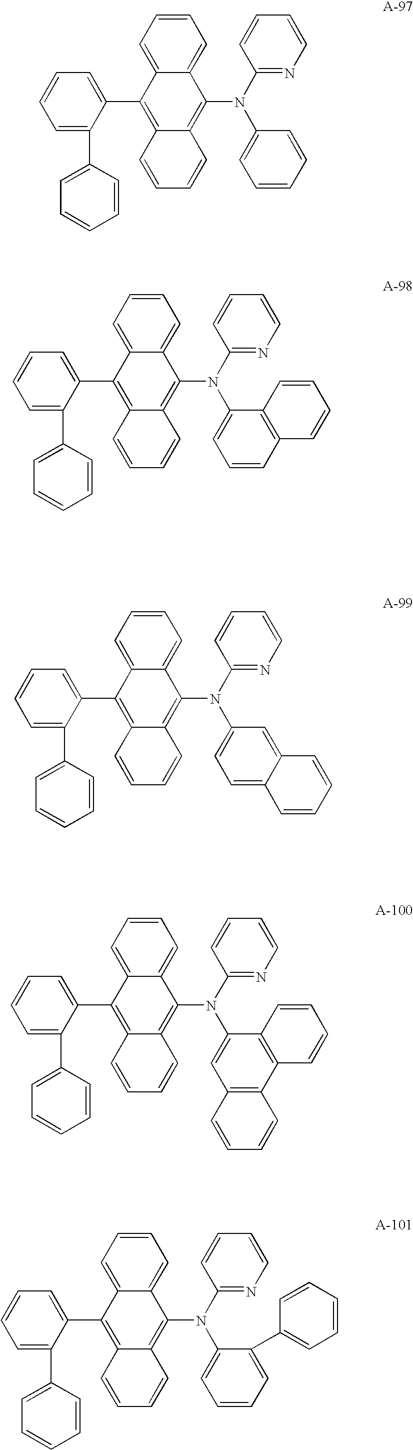

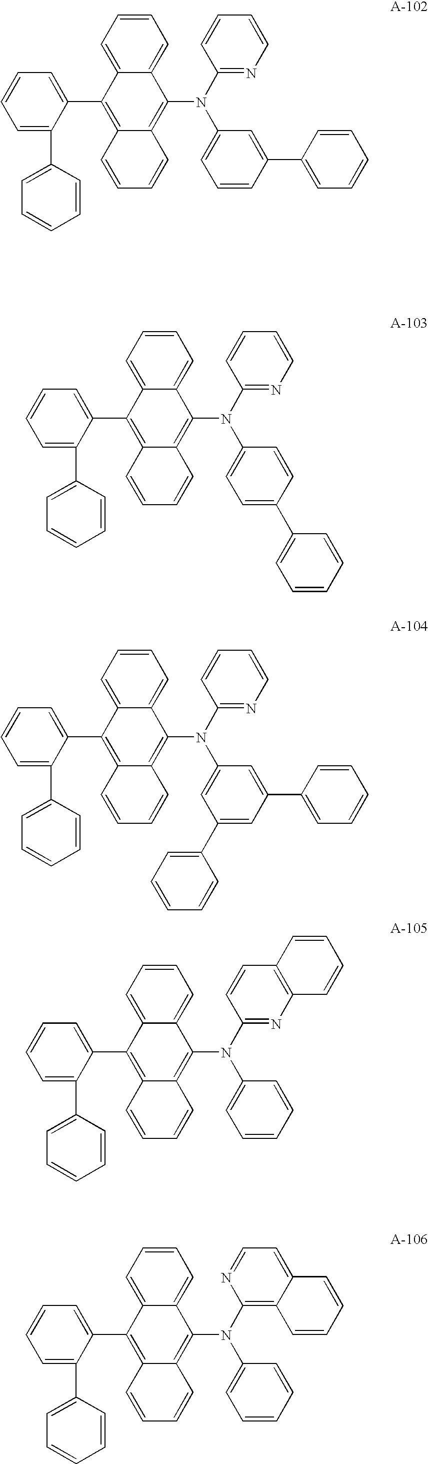

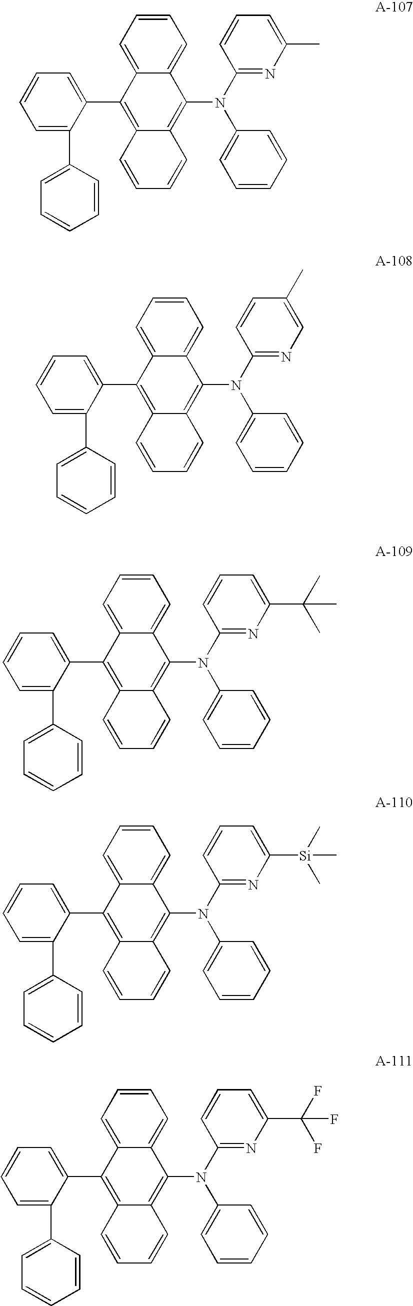

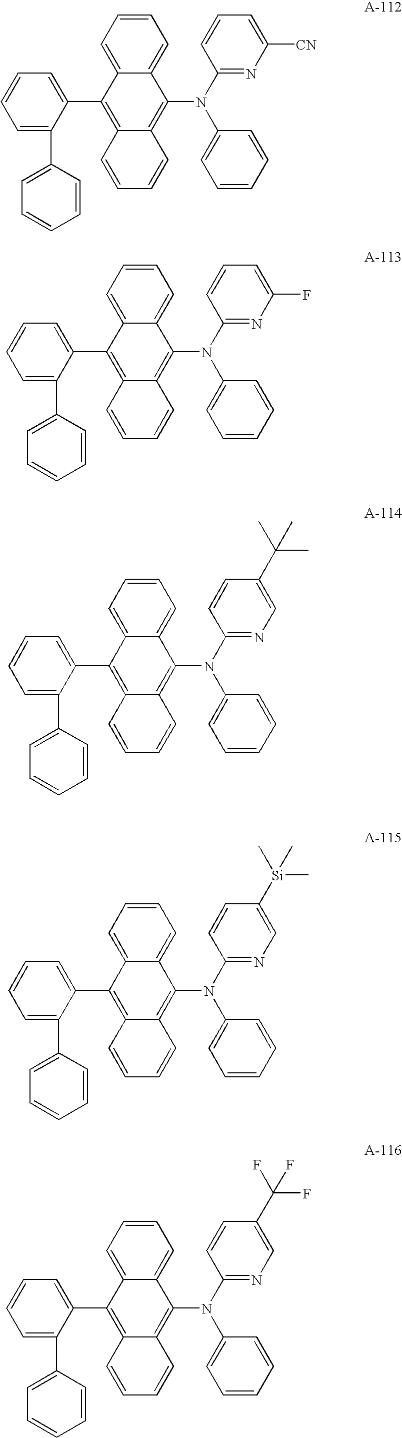

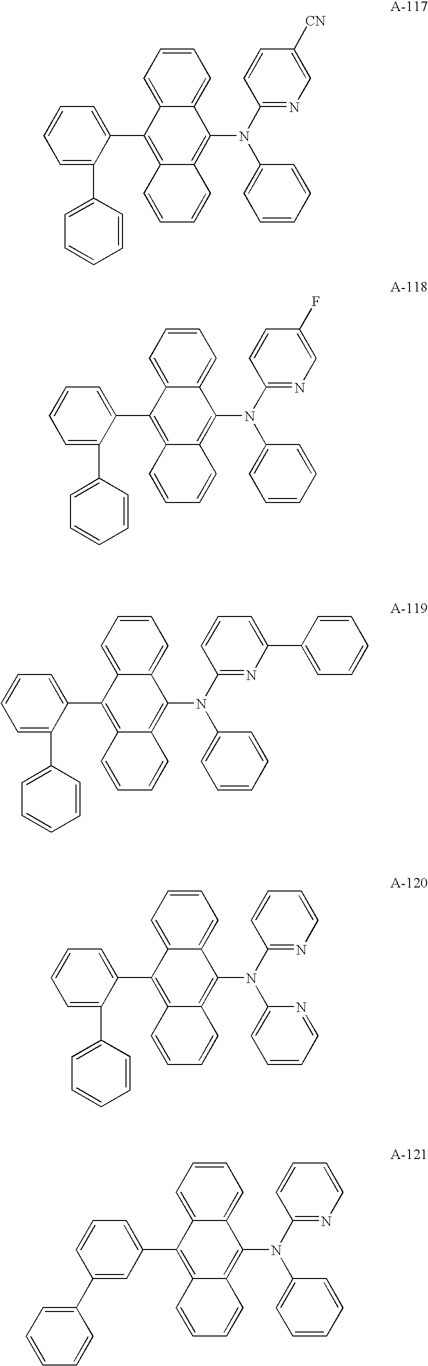

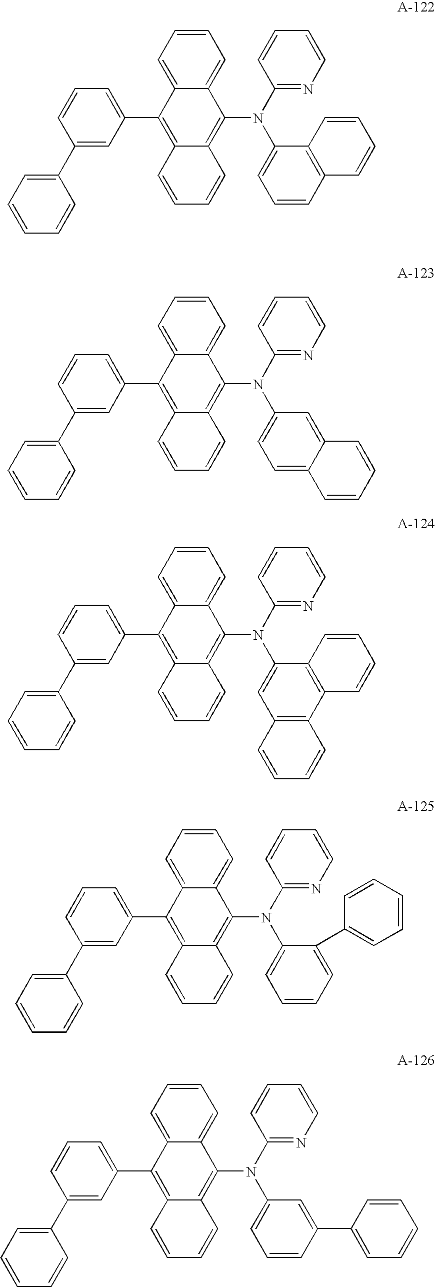

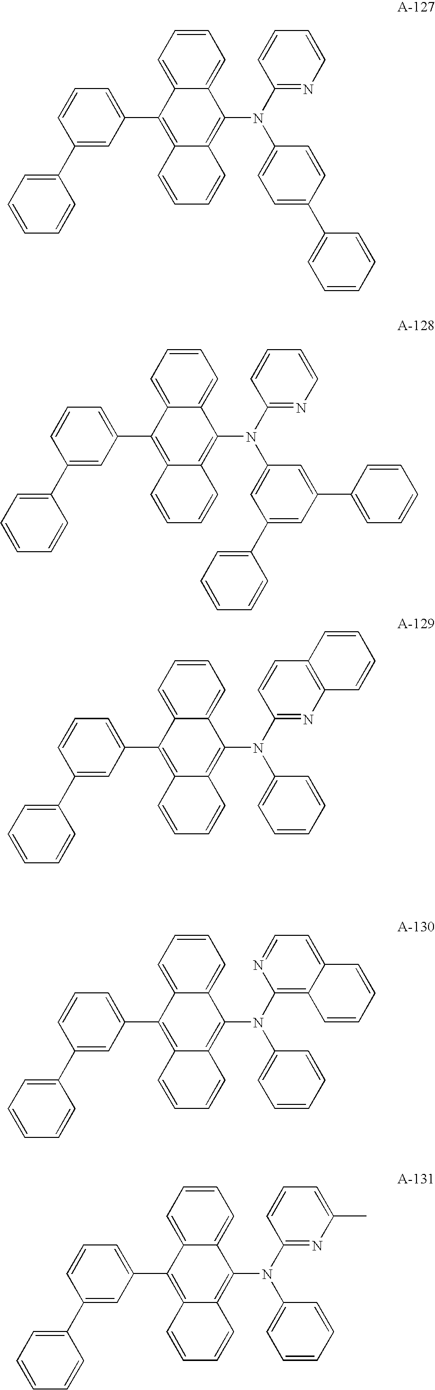

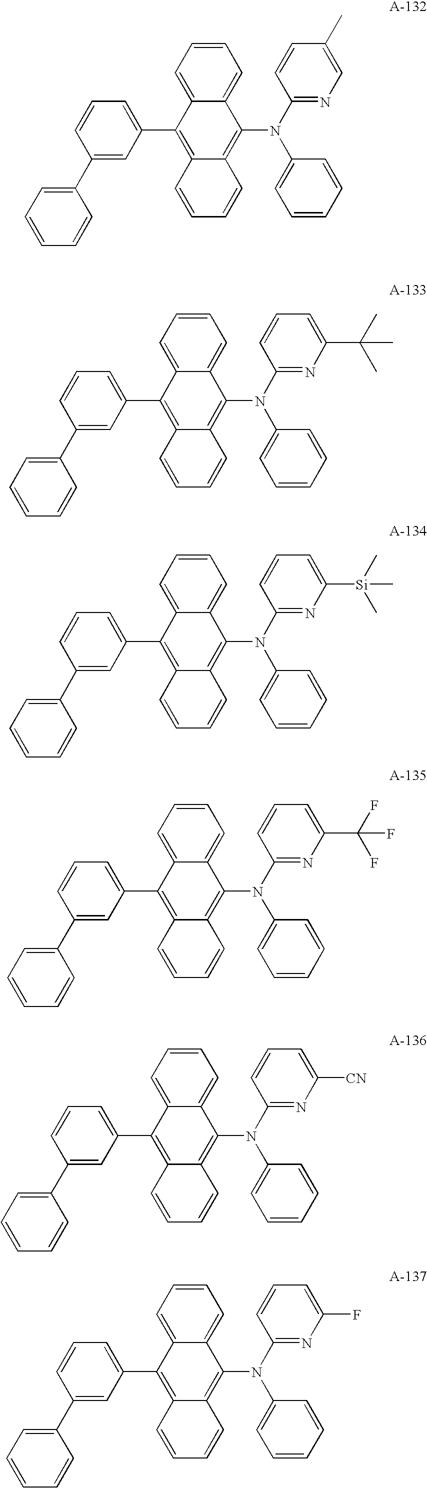

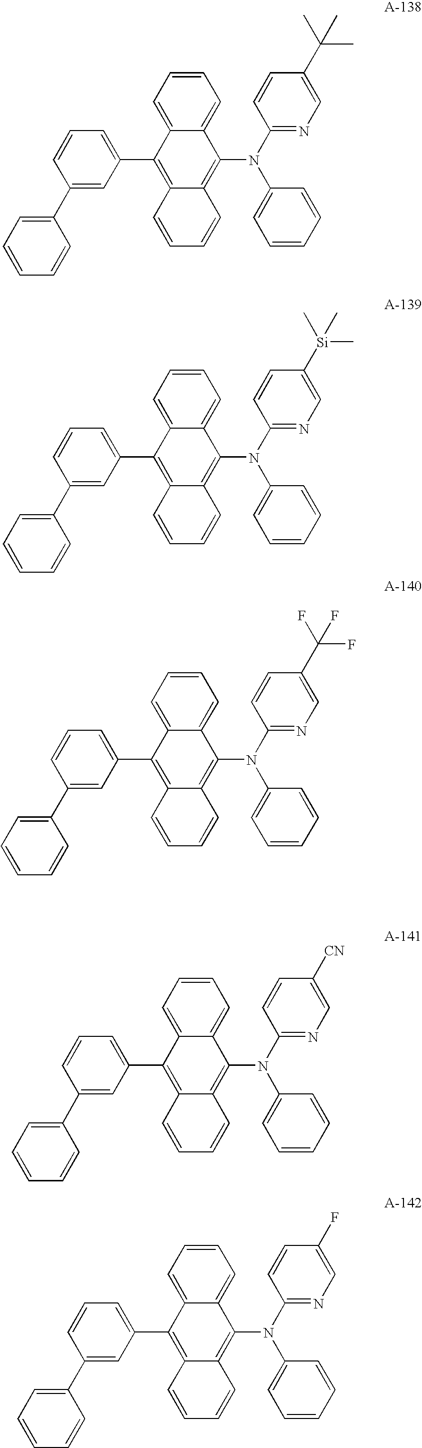

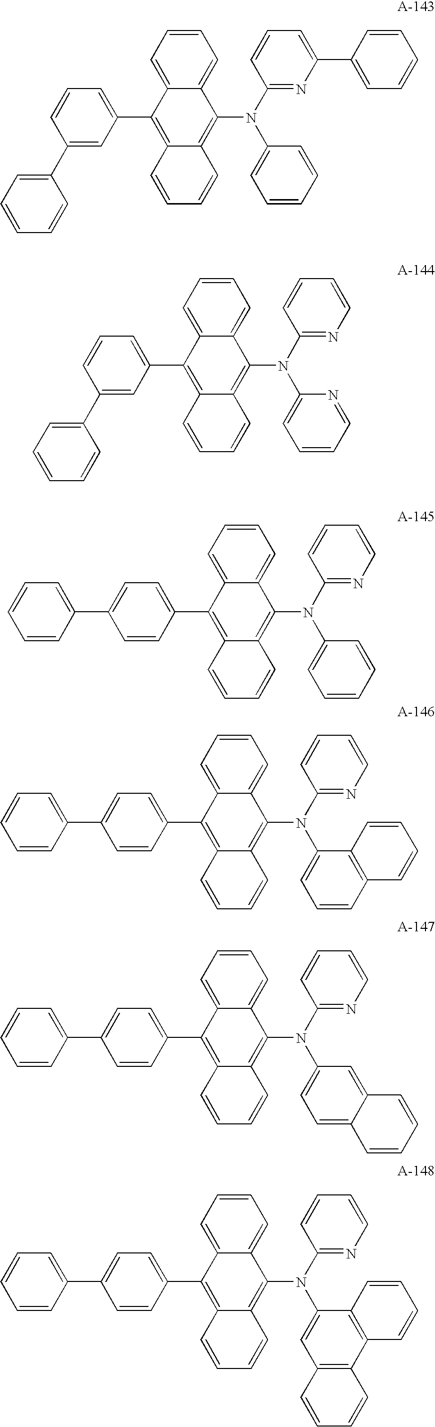

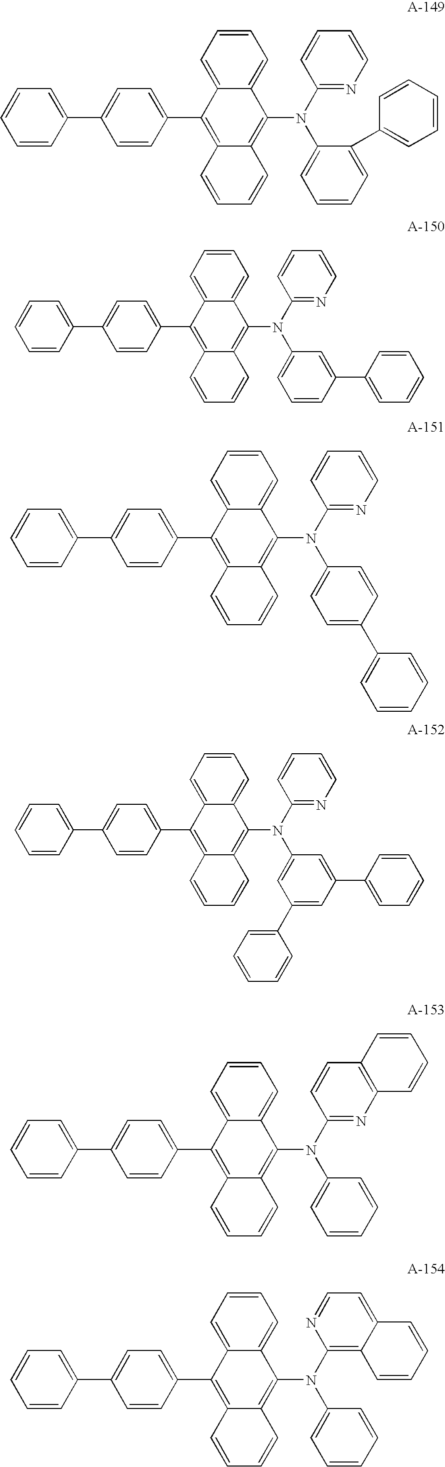

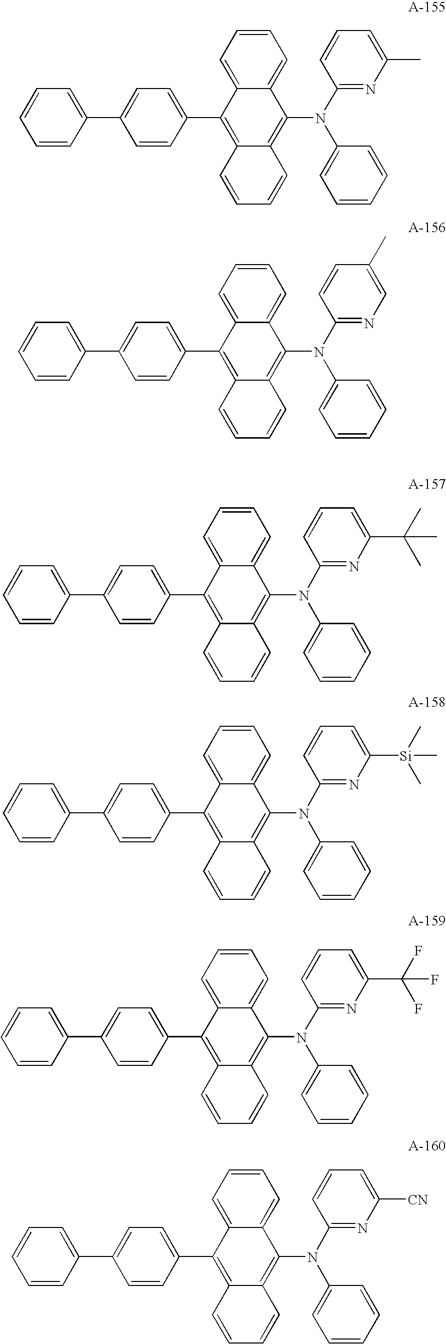

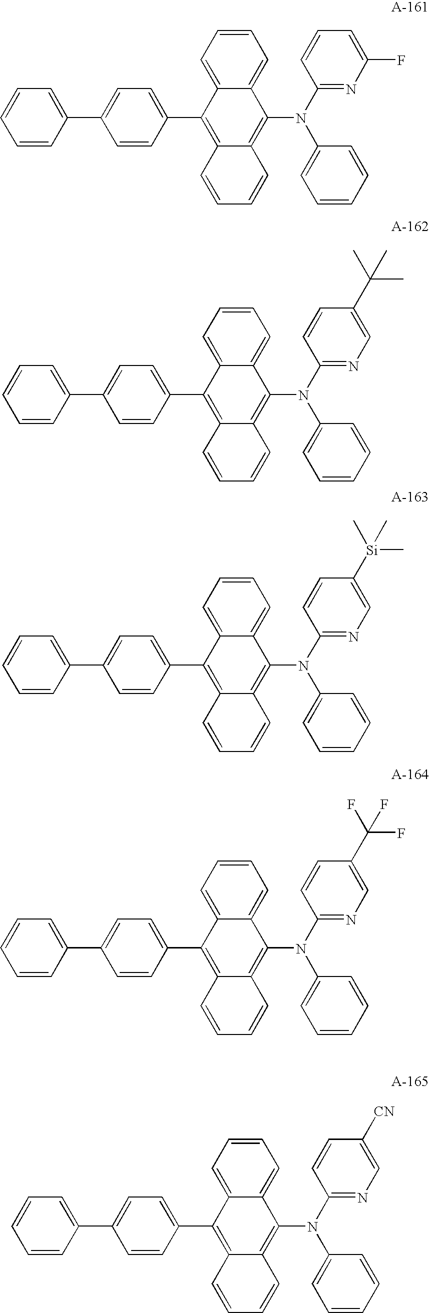

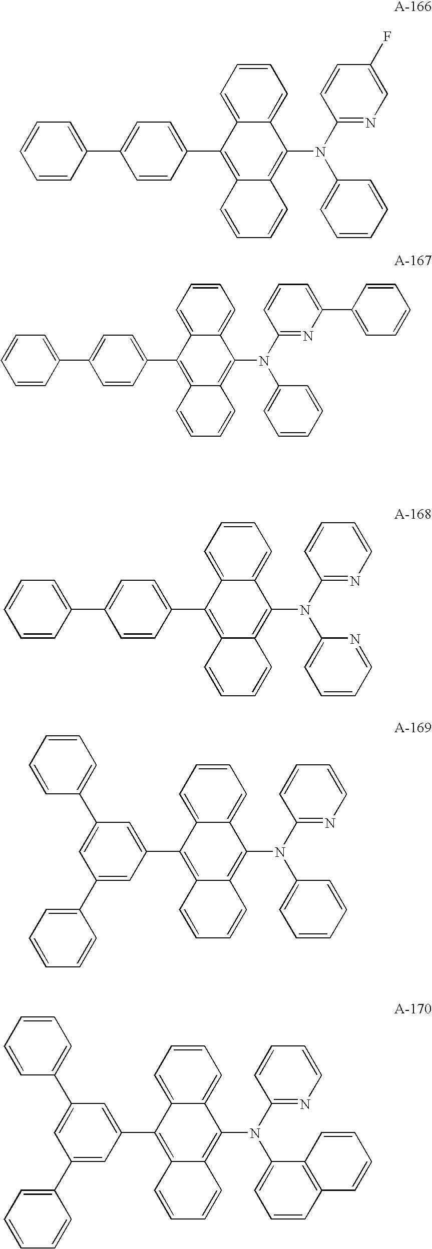

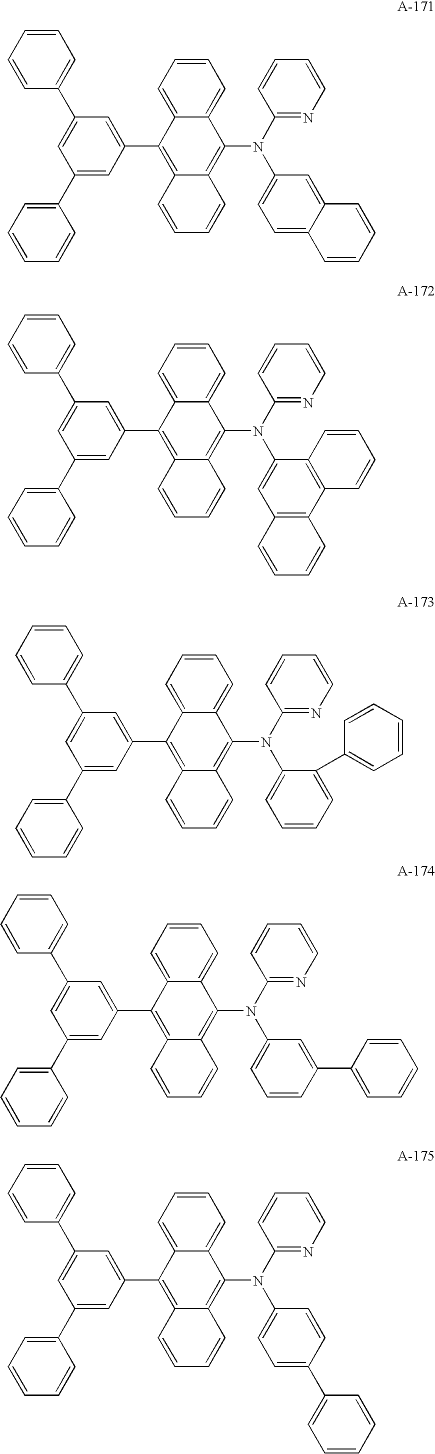

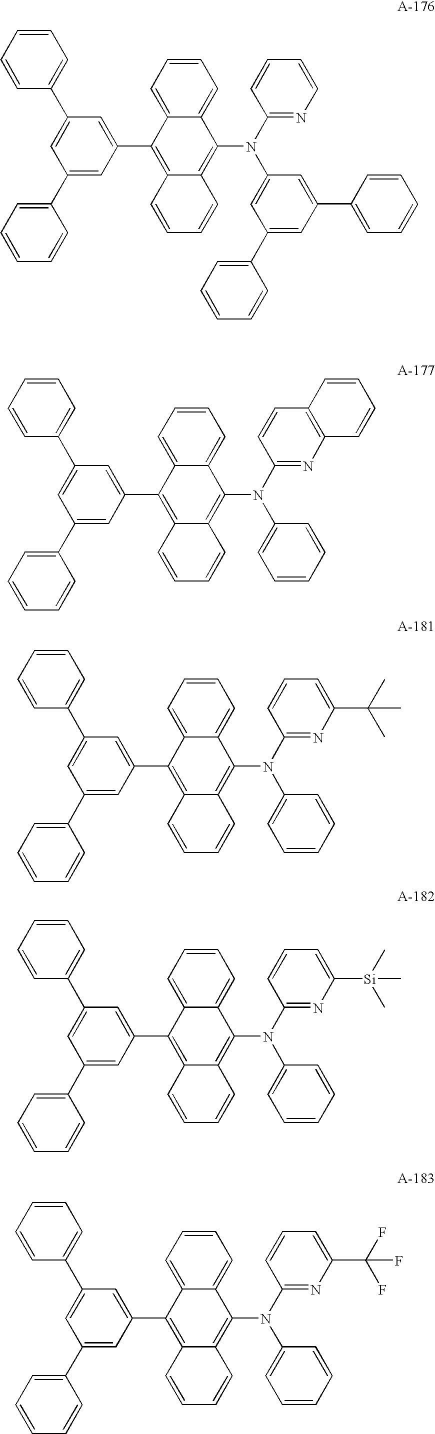

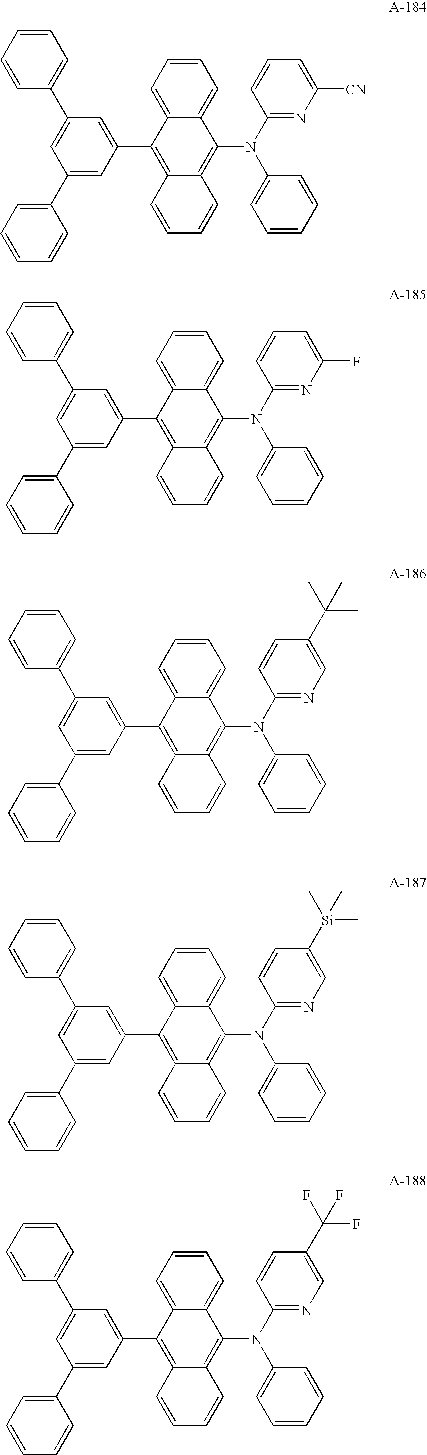

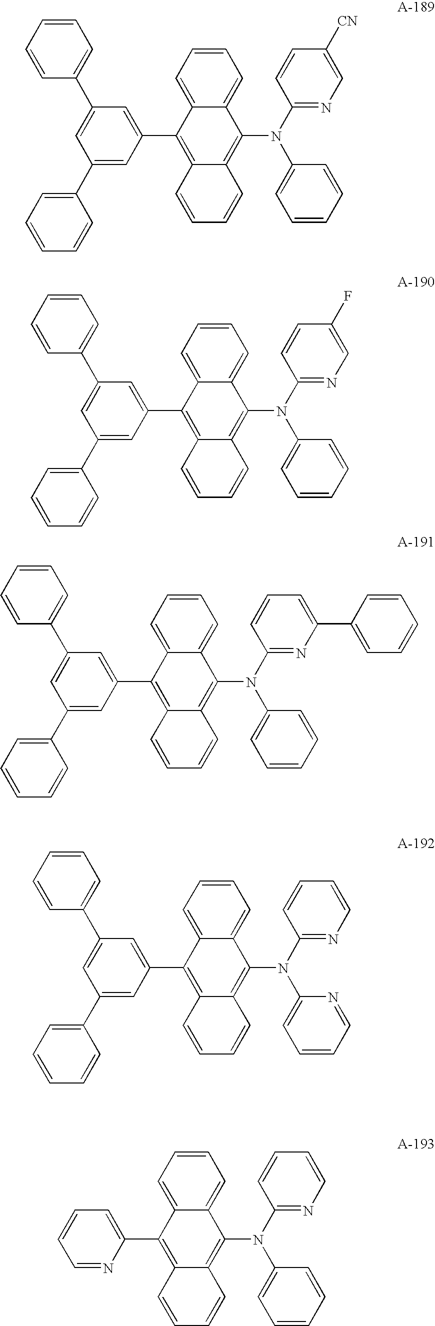

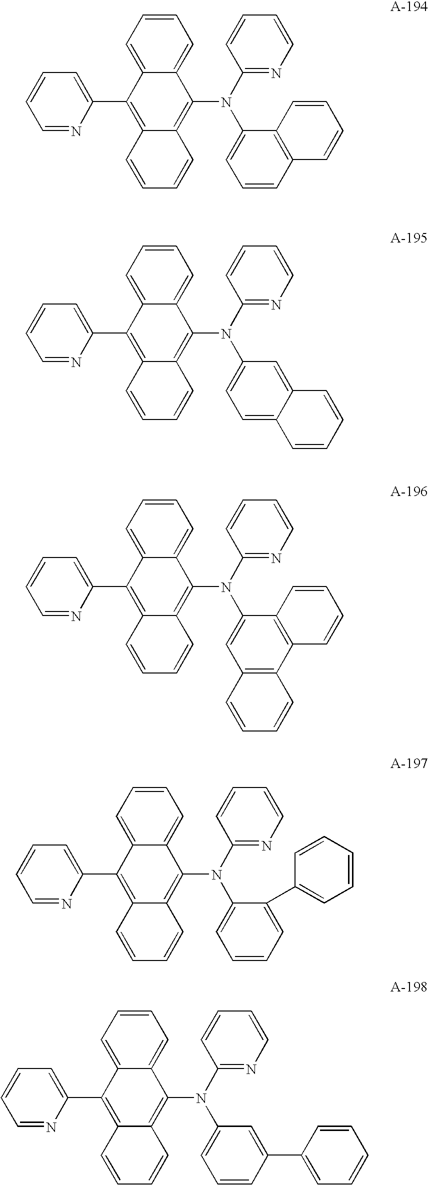

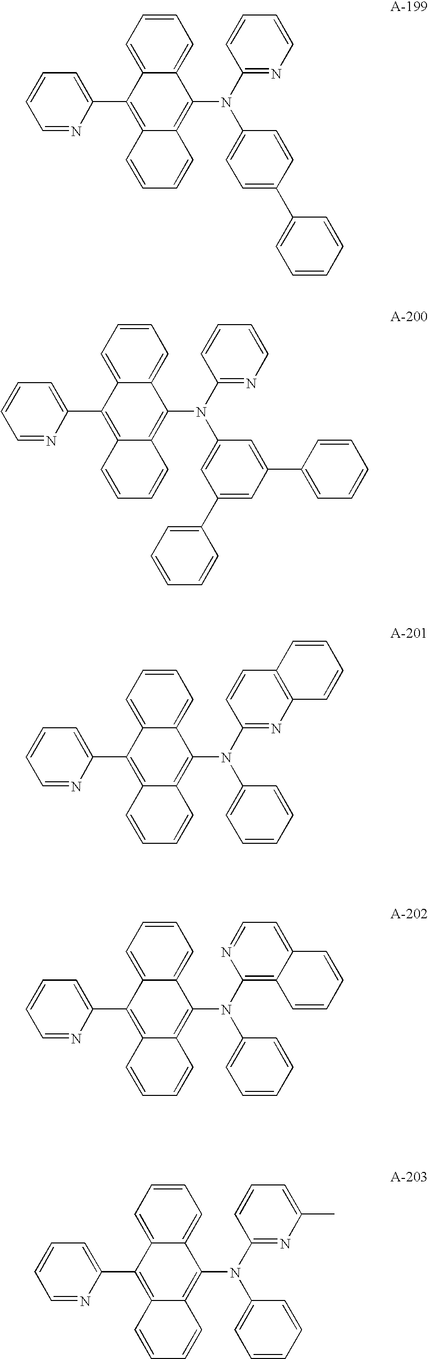

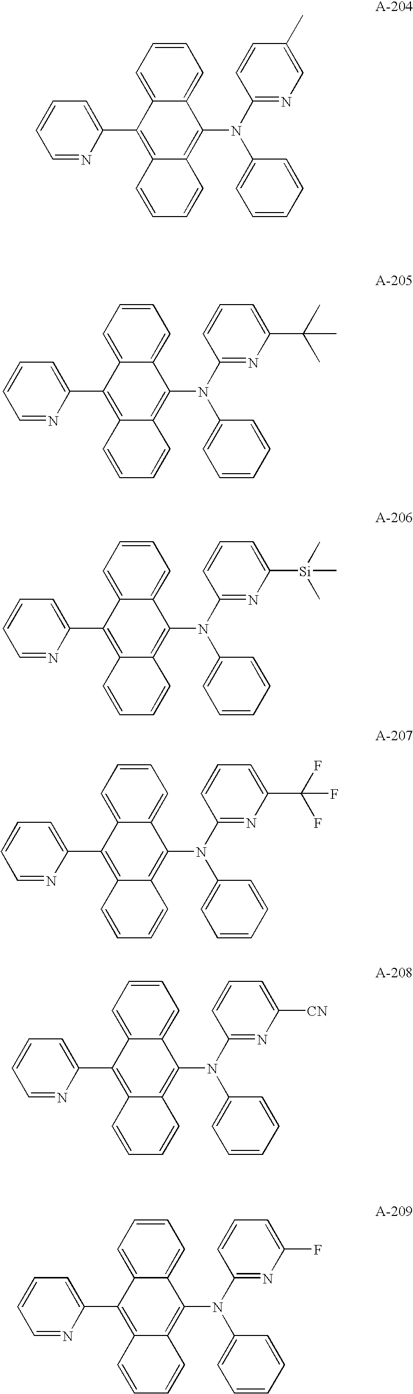

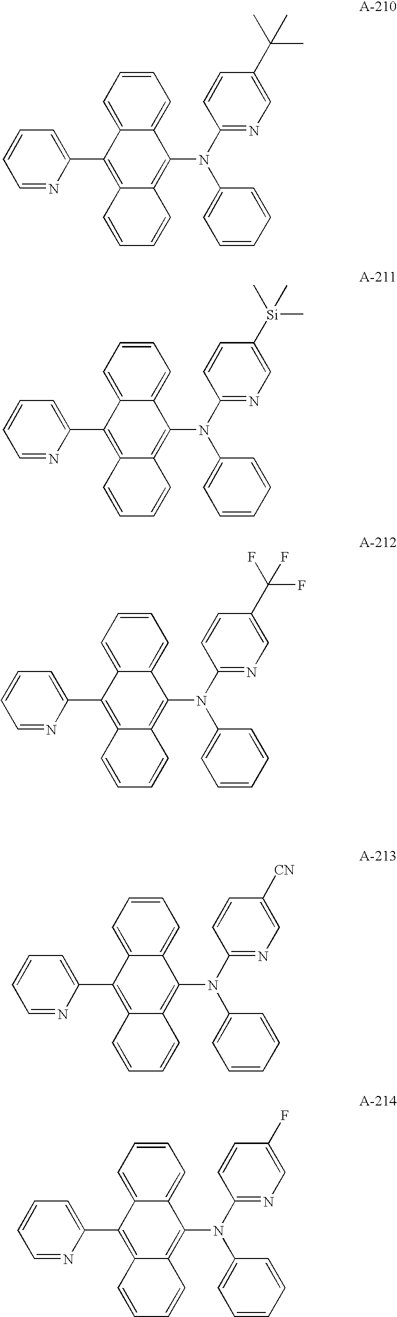

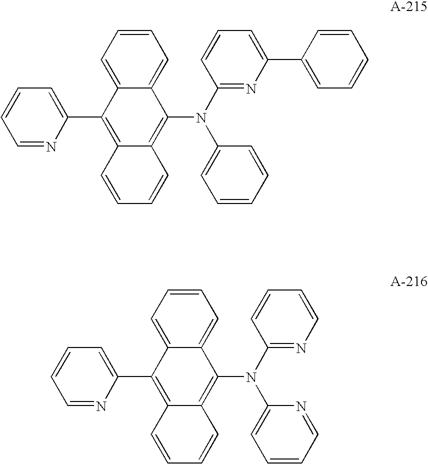

### Synthesis

A synthesis example of the electron transporting-injection compound marked by A-25 in the above Formula 2 is explained. The A-25 electron transporting-injection compound is 9-naphthyl-10-(phenyl-2-pyridyl)amineanthracene.

### 1. Synthesis of phenyl-2-pyridylamine

phenyl-2-pyridylamine is synthesized by following Reaction Formula 1.

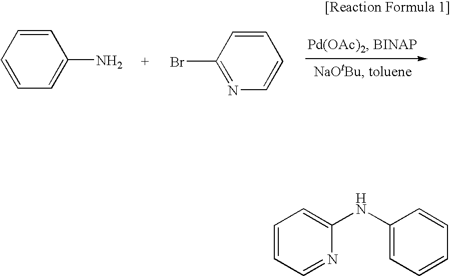

Aniline (5 g, 0.05 mol), 2-bromopyridine (8.5 g, 0.05 mol), palladium acetate (0.04 g, 0.16 mmol), BINAP (2,2′-bis(diphenylphosphino)-1,1′-binaphthyl) (0.13 g, 0.21 mmol), and NaOtBu (7.6 g, 0.08 mol) are put in a two-neck round-bottom flask and dissolved in toluene (80 mL). Subsequently, the resulting solution is refluxed for 12 hours. After completion of the reaction, the solution is cooled to a room temperature, and toluene is evaporated. Methanol (20 mL) is added thereto, and the resulting residence is filtered. Next, by re-crystallizing and filtering with methylene chloride and methanol, phenyl-2-pyridylamine (6.3 g, yield: 70%) is yield.

### 2. Synthesis of 9-bromo-10-(phenyl-2-pyridyl)amineanthracene

9-bromo-10-(phenyl-2-pyridyl)amineanthracene is synthesized by following Reaction Formula 2.

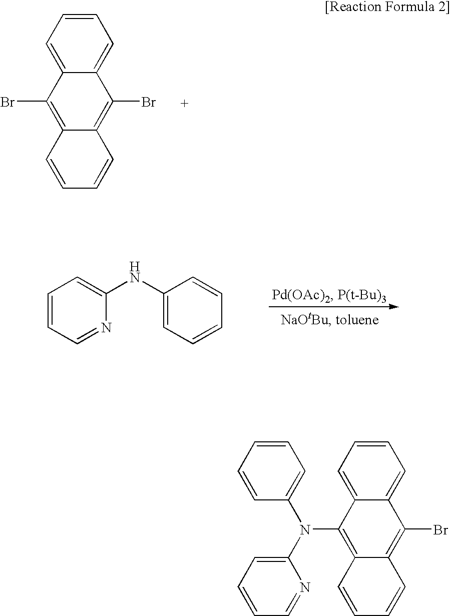

9,10-dibromoanthracene (2 g, 5.9 mmol), phenyl-2-pyridylamine (1.0 g, 5.9 mmol), palladium acetate (0.04 g, 0.16 mmol), tert-butylphosphine (0.03 g, 0.21 mmol), and NaOtBu (1.76 g, 17.9 mmol) are put in a two-neck round-bottom flask and dissolved in toluene (40 mL). Subsequently, the resulting solution is refluxed for 12 hours. After completion of the reaction, the solution is cooled to a room temperature, and toluene is evaporated. Methanol (20 mL) is added thereto, and the resulting residence is filtered. Next, by re-crystallizing and filtering with methylene chloride and methanol, 9-bromo-10-(phenyl-2-pyridyl)amineanthracene (1.8 g, yield: 70%) is yield.

### 3. Synthesis of 9-naphthyl-10-(phenyl-2-pyridyl)amineanthracene

9-naphthyl-10-(phenyl-2-pyridyl)amineanthracene is synthesized by following Reaction Formula 3.

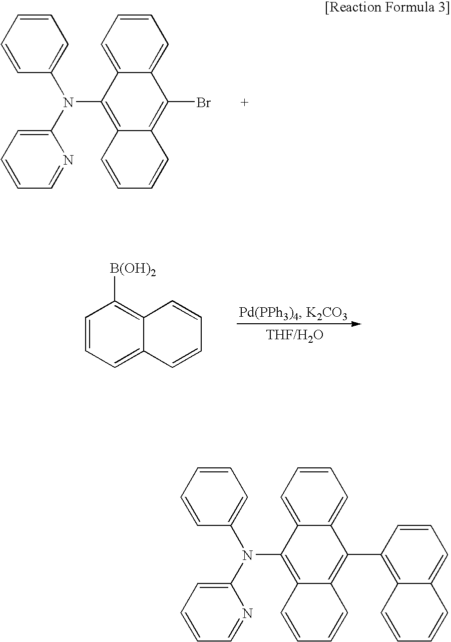

9-bromo-10-(phenyl-2-pyridyl)amineanthracene (2 g, 4.7 mmol), 1-naphthyl-boronic acid (1 g, 5.2 mmol), tetrakis(triphenylphosphine)palladium(0) (Pd(PPh₃)₄ (0.1 g, 0.9 mmol), and a solution (80 mL) of 2M-K₂CO₃ and tetrahydrofuran (THF), where a ratio of 2M-K₂CO₃ to THF is 1:1, are put in a two-neck round-bottom flask and refluxed for 12 hours. After completion of the reaction, the resulting solution is cooled to a room temperature and extracted by using methylene chloride. The solvent is evaporated, and then being refined through a silica gel column to yield 9-naphthyl-10-(phenyl-2-pyridyl)amineanthracene (1.5 g, yield: 70%).

Hereinafter, a detailed description will be made of preferred examples associated with the OELD according to the present invention. More specifically, the examples relate to an OELD including the electron transporting-injection compound of Formula 1 as an electron transporting-injection layer. In following Examples 1 to 4, lithium fluoride (LiF) is used as an electron injection layer. Alternatively, the electron transporting-injection compound according to the present invention can be used as both an electron injection layer and an electron transporting layer without the LiF layer.

### EXAMPLES

### Example 1

An indium-tin-oxide (ITO) layer is patterned on a substrate and washed such that an emission area of the ITO layer is 3 mm*3 mm. The substrate is loaded in a vacuum chamber, and the process pressure is adjusted to 1*10⁻⁶ ton. CuPC (about 650 angstroms) represented by following Formula 3-1, 4,4′-bis[N-(1-naphtyl)-N-phenylamino]-biphenyl (NPD) (about 400 angstroms) represented by following Formula 3-2, an emitting layer (about 200 angstroms) including DPVBi, which is represented by following Formula 3-3, as a host and a compound, which is represented by following Formula 3-4, as a dopant (about 1 weight %), an electron transporting-injection compound represented by A-01 in the above Formula 2 (about 350 angstroms), lithium fluoride (LiF) (about 5 angstroms) and aluminum (Al) (about 1000 angstroms) are sequentially formed on the ITO layer such that an OELD is fabricated.

The OELD produces a brightness of 779 cd/m² at an electric current of 0.9 mA and a voltage of 5.4 V. At this time, the X index and Y index of CIE color coordinates are 0.136 and 0.189, respectively.

### Example 2

An indium-tin-oxide (ITO) layer is patterned on a substrate and washed such that an emission area of the ITO layer is 3 mm*3 mm. The substrate is loaded in a vacuum chamber, and the process pressure is adjusted to 1*10⁻⁶ ton. CuPC (about 650 angstroms) represented by following Formula 3-1, 4,4′-bis[N-(1-naphtyl)-N-phenylamino]-biphenyl (NPD) (about 400 angstroms) represented by following Formula 3-2, an emitting layer (about 200 angstroms) including DPVBi, which is represented by following Formula 3-3, as a host and a compound, which is represented by following Formula 3-4, as a dopant (about 1 weight %), an electron transporting-injection compound represented by A-10 in the above Formula 2 (about 350 angstroms), lithium fluoride (LiF) (about 5 angstroms) and aluminum (Al) (about 1000 angstroms) are sequentially formed on the ITO layer such that an OELD is fabricated.

The OELD produces a brightness of 765 cd/m² at an electric current of 0.9 mA and a voltage of 5.5 V. At this time, the X index and Y index of CIE color coordinates are 0.132 and 0.180, respectively.

### Example 3

An indium-tin-oxide (ITO) layer is patterned on a substrate and washed such that an emission area of the ITO layer is 3 mm*3 mm. The substrate is loaded in a vacuum chamber, and the process pressure is adjusted to 1*10⁻⁶ ton. CuPC (about 650 angstroms) represented by following Formula 3-1, 4,4′-bis[N-(1-naphtyl)-N-phenylamino]-biphenyl (NPD) (about 400 angstroms) represented by following Formula 3-2, an emitting layer (about 200 angstroms) including DPVBi, which is represented by following Formula 3-3, as a host and a compound, which is represented by following Formula 3-4, as a dopant (about 1 weight %), an electron transporting-injection compound represented by A-11 in the above Formula 2 (about 350 angstroms), lithium fluoride (LiF) (about 5 angstroms) and aluminum (Al) (about 1000 angstroms) are sequentially formed on the ITO layer such that an OELD is fabricated.

The OELD produces a brightness of 755 cd/m² at an electric current of 0.9 mA and a voltage of 5.4 V. At this time, the X index and Y index of CIE color coordinates are 0.135 and 0.190, respectively.

### Example 4

An indium-tin-oxide (ITO) layer is patterned on a substrate and washed such that an emission area of the ITO layer is 3 mm*3 mm. The substrate is loaded in a vacuum chamber, and the process pressure is adjusted to 1*10⁻⁶ ton. CuPC (about 650 angstroms) represented by following Formula 3-1, 4,4′-bis[N-(1-naphtyl)-N-phenylamino]-biphenyl (NPD) (about 400 angstroms) represented by following Formula 3-2, an emitting layer (about 200 angstroms) including DPVBi, which is represented by following Formula 3-3, as a host and a compound, which is represented by following Formula 3-4, as a dopant (about 1 weight %), an electron transporting-injection compound represented by A-15 in the above Formula 2 (about 350 angstroms), lithium fluoride (LiF) (about 5 angstroms) and aluminum (Al) (about 1000 angstroms) are sequentially formed on the ITO layer such that an OELD is fabricated.

The OELD produces a brightness of 730 cd/m² at an electric current of 0.9 mA and a voltage of 5.8 V. At this time, the X index and Y index of CIE color coordinates are 0.138 and 0.200, respectively.

### Comparative Example 1

An indium-tin-oxide (ITO) layer is patterned on a substrate and washed such that an emission area of the ITO layer is 3 mm*3 mm. The substrate is loaded in a vacuum chamber, and the process pressure is adjusted to 1*10⁻⁶ torr. CuPC (about 650 angstroms) represented by following Formula 3-1, 4,4′-bis[N-(1-naphtyl)-N-phenylamino]-biphenyl (NPD) (about 400 angstroms) represented by following Formula 3-2, an emitting layer (about 200 angstroms) including DPVBi, which is represented by following Formula 3-3, as a host and a compound, which is represented by following Formula 3-4, as a dopant (about 1 weight %), Alq3 (about 350 angstroms) represented by following Formula 3-5, lithium fluoride (LiF) (about 5 angstroms) and aluminum (Al) (about 1000 angstroms) are sequentially formed on the ITO layer such that an OELD is fabricated.

The OELD produces a brightness of 655 cd/m² at an electric current of 0.9 mA and a voltage of 6.4 V. At this time, the X index and Y index of CIE color coordinates are 0.136 and 0.188, respectively.

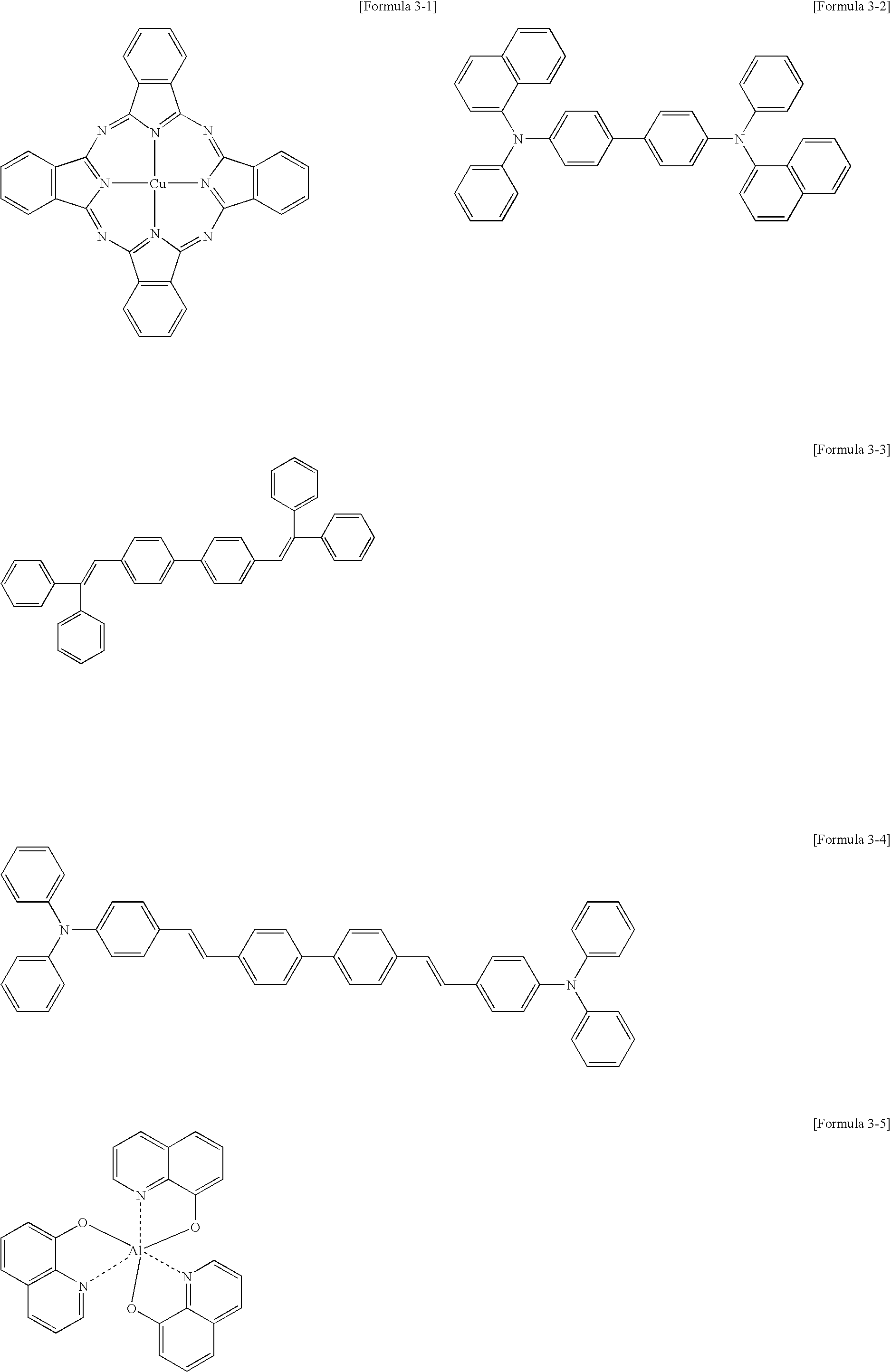

The OELD fabricated in Examples 1 to 4 and Comparative Example 1 is evaluated for efficiency, brightness, and so on. A voltage has a dimension of [V], an electric current has a dimension of [mA], a brightness has a dimension of [cd/m²], a current efficiency has a dimension of [cd/A], and a power efficiency has a dimension of [1 m/W]. The evaluated results are shown in Table 1.

TABLE 1

| | | Electric | Bright- | Current | Power | CIE | CIE |
|---|---|---|---|---|---|---|---|
| | voltage | current | ness | efficiency | efficiency | (X) | (Y) |
| Ex. 1 | 5.4 | 0.9 | 779 | 7.8 | 4.53 | 0.136 | 0.189 |
| Ex. 2 | 5.5 | 0.9 | 765 | 7.6 | 4.34 | 0.132 | 0.180 |
| Ex. 3 | 5.4 | 0.9 | 755 | 7.5 | 4.36 | 0.135 | 0.190 |
| Ex. 4 | 5.8 | 0.9 | 730 | 7.3 | 3.95 | 0.138 | 0.200 |
| Com. | 6.7 | 0.9 | 526 | 5.26 | 2.47 | 0.136 | 0.188 |
| Ex. 1 | | | | | | | |

As shown in Table 1, the OELD in Examples 1 to 4 has improved luminescent efficiency such that power consumption for the OELD is reduced. As a result, a lifetime of the OELD using the electron transporting-injection compound according to the present invention is improved.

### Second Embodiment

An electron transporting-injection compound according to the second embodiment of the present invention includes an asymmetric anthracene structure. In more detail, one side position of the anthracene is substituted by an aniline group, which is substituted by one of substituted or non-substituted aromatic group, substituted or non-substituted heterocyclic group, or of substituted or non-substituted aliphatic group, and the other side position of the anthracene is substituted by one of substituted or non-substituted aromatic group, substituted or non-substituted heterocyclic group, or of substituted or non-substituted aliphatic group. Namely, the one side position of the anthracene is substituted by a phenyl including an ammonium salt. As a result, an organic electroluminescent diode including the electron transporting-injection compound according to the second embodiment of the present invention can have high luminescent efficiency, low driving voltage and long lifetime.

The electron transporting-injection compound according to the second embodiment of the present invention is represented by following Formula 4.

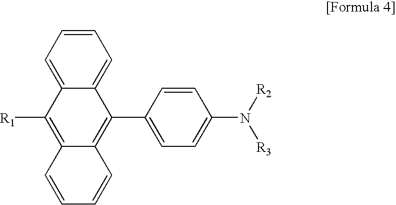

In the above Formula 4, each of R1, R2, and R3 is selected from substituted or non-substituted aromatic group, substituted or non-substituted heterocyclic group, or of substituted or non-substituted aliphatic group, and at least one of R2 and R3 is selected from substituted or non-substituted heterocyclic group.

In addition, the substituted or non-substituted heterocyclic group for at least one of R2 and R3 is pyridyl, and the electron transporting-injection compound according to the second embodiment has a following structure.

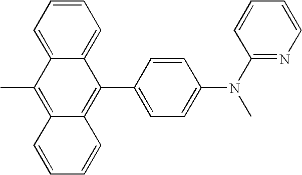

Since the compound is substituted by pyridyl to have a structure of

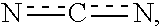

an electron attraction strength is increased such that the electron transporting-injection compound according to the present invention has improved properties for transporting and injecting an electron. As a result, luminescent efficiency is improved. The electron transporting-injection compound has an amorphous property due to an asymmetric structure such that a property of film is improved. In addition, a benzene ring of the aniline group is positioned between the anthracene and an ammonium salt of the aniline such that an electron attraction property is increased and a lifetime is improved due to a steric hindrance. Moreover, since a luminance property of a blue emitting layer is strongly affected by properties of an electron transporting layer, the blue emitting layer can produce a deep blue color due to the benzene ring.

For example, the aromatic group includes phenyl, byphenyl, naphthyl, phenanthrenyl, and terphenyl, and the heterocyclic group includes pyridyl, bipyridyl, phenylpyridyl, pyridylphenyl, terpyridyl, quinolinyl, isoquinolinyl, and quinoxalinyl. The aliphatic group includes methyl, ethyl, propyl, isopropyl, butyl, and tert-butyl.

A substituent for each of R1, R2 and R3 is one of aryl, alkyl, alkoxy, allyamino, alkylamino, amino, halogen and cyano. For example, the substituent for each of R1, R2 and R3 is one of methyl, ethyl, propyl, isopropyl, tert-butyl, methoxy, ethoxy, butoxy, trimethylsilyl, fluorine and chlorine.

When at least one of R1, R2 and R3 is substituted by naphthyl, such as

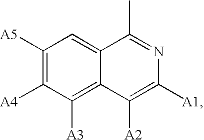

at least one of A1 to A5 is methyl. Alternatively, when at least one of R1, R2 and R3 is substituted by naphthyl, such as

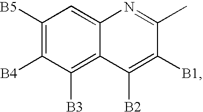

at least one of B1 to B5 is methyl. When the electron transporting-injection compound includes naphthyl substituted at least one methyl, luminescent efficiency and lifetime is further improved.

For example, the electron transporting-injection compound represented by Formula 4 is one of compounds in following Formula 5. For convenience, B-01 to B-216 are respectively marked to compounds.

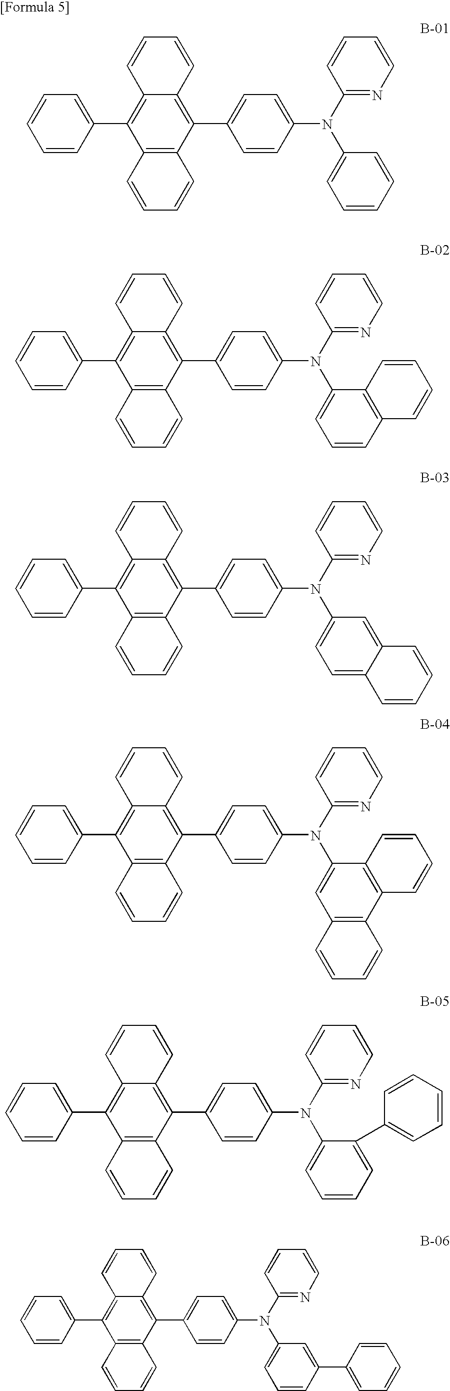

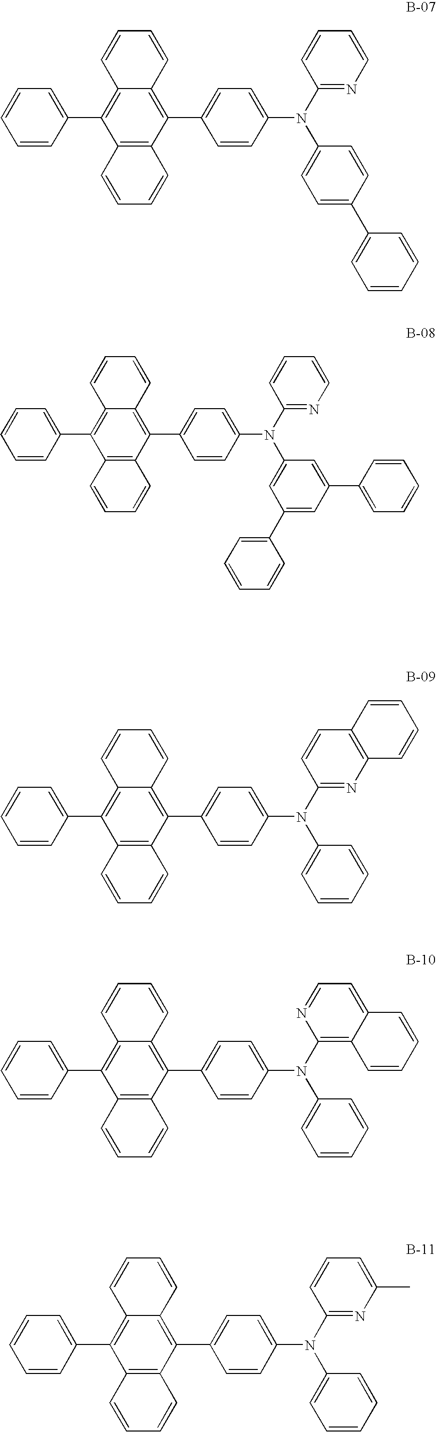

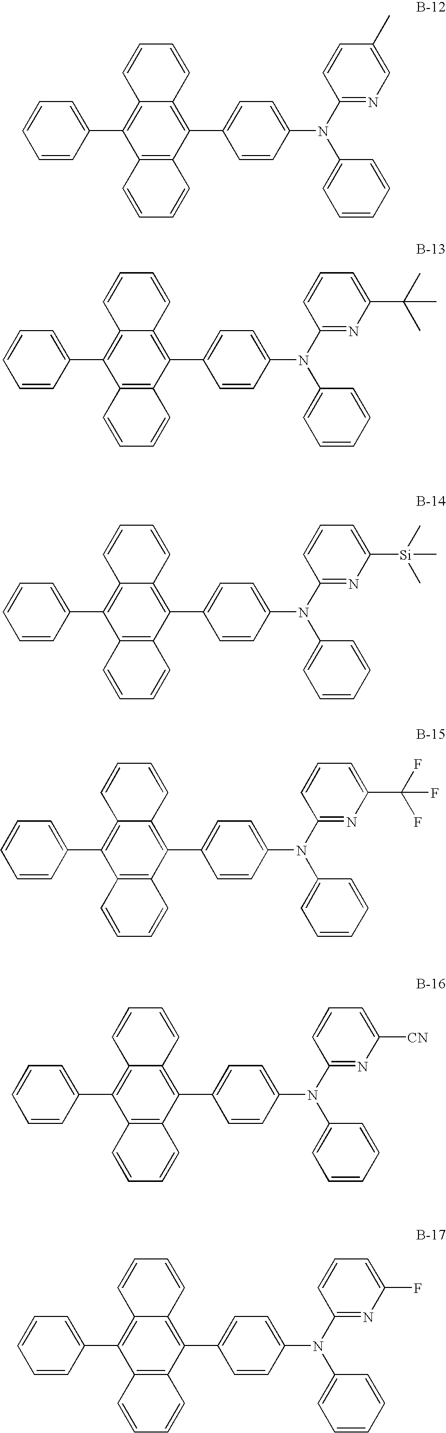

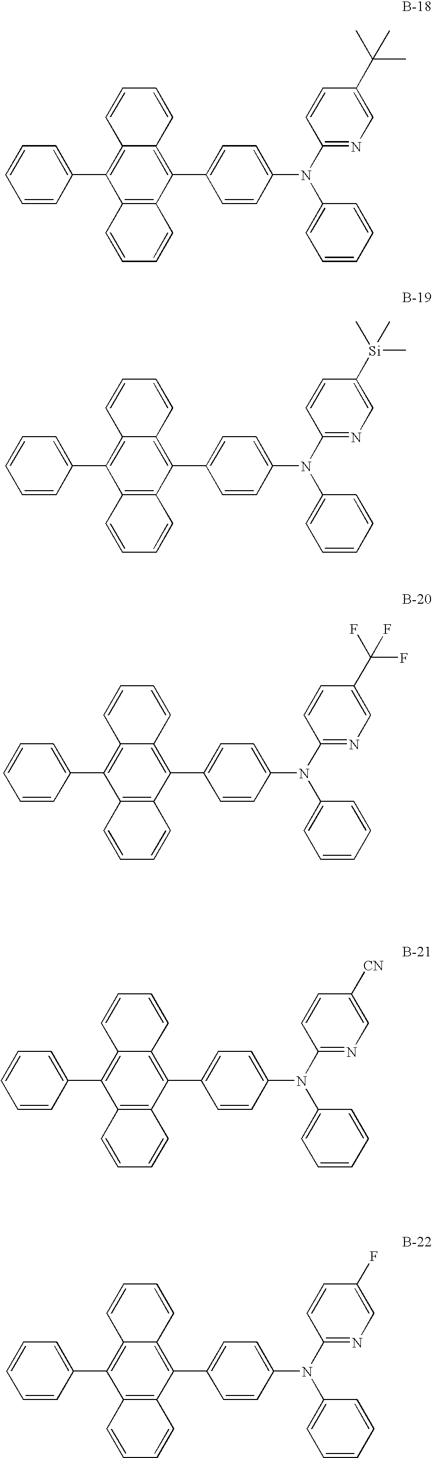

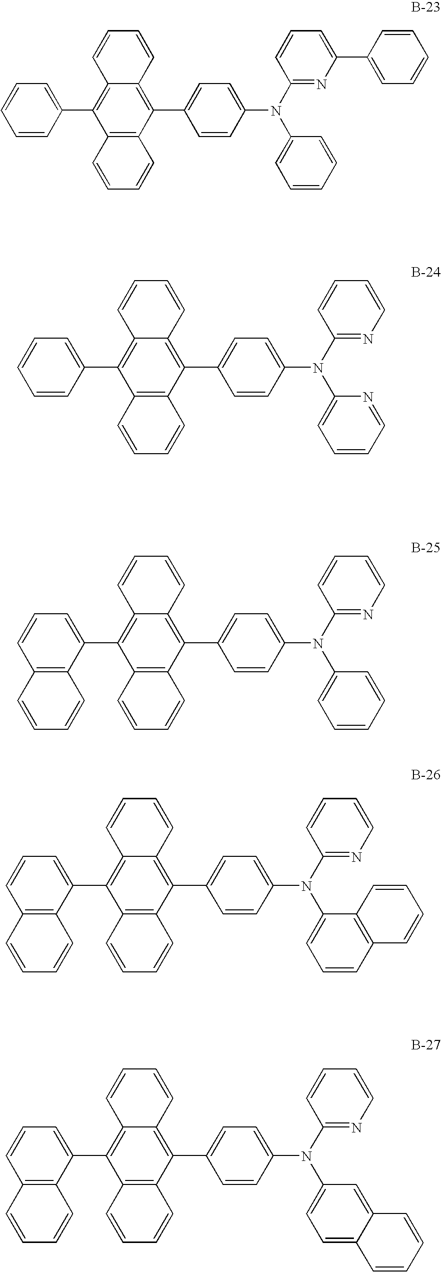

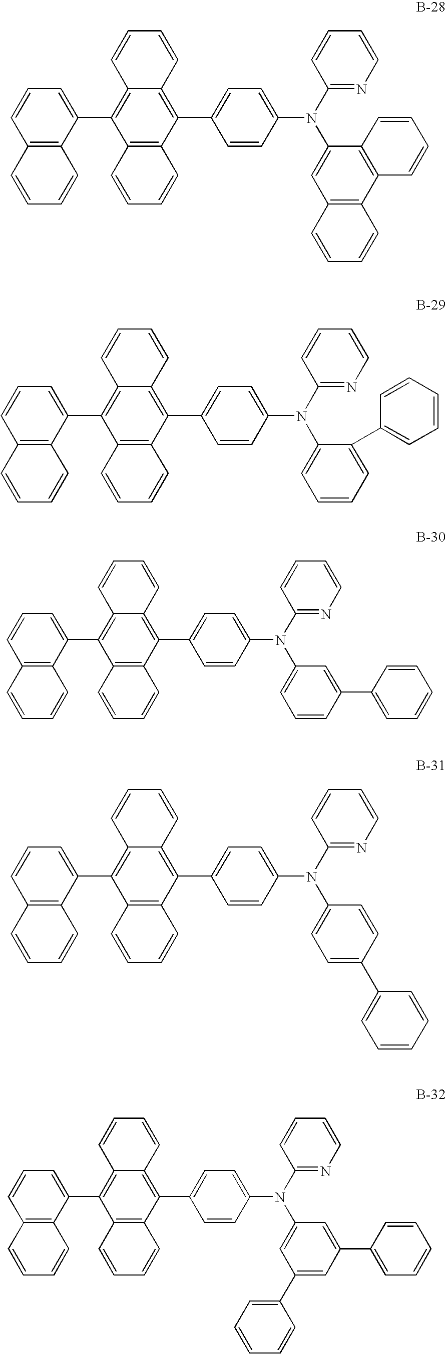

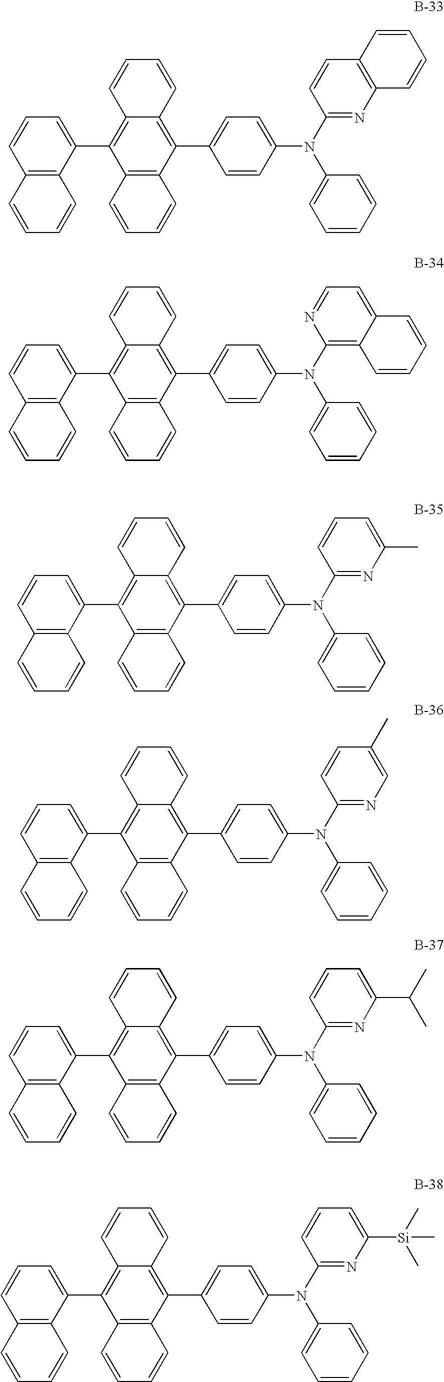

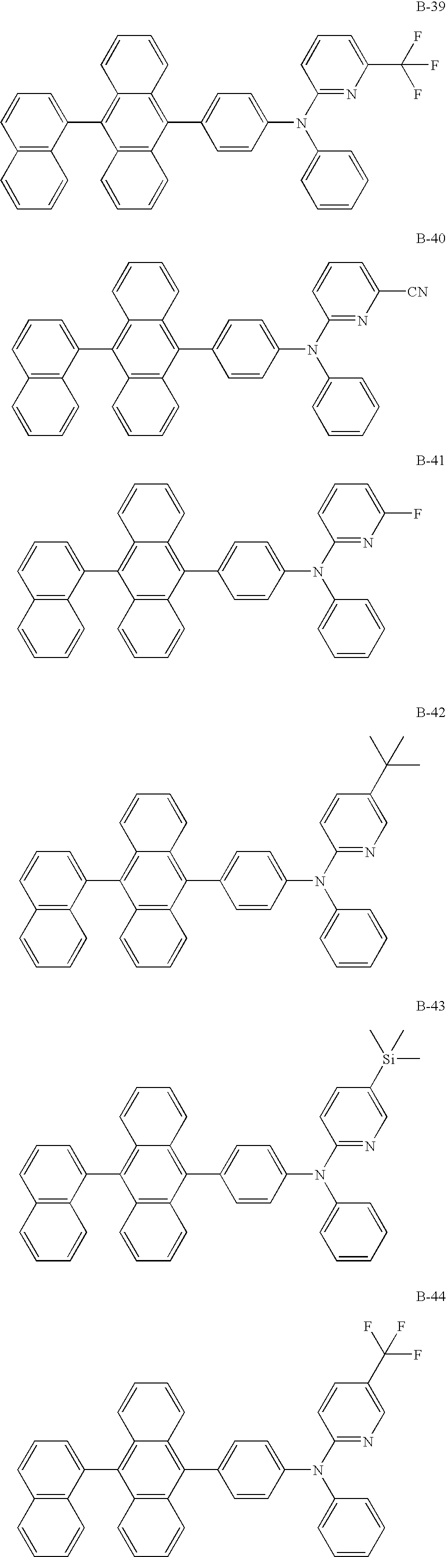

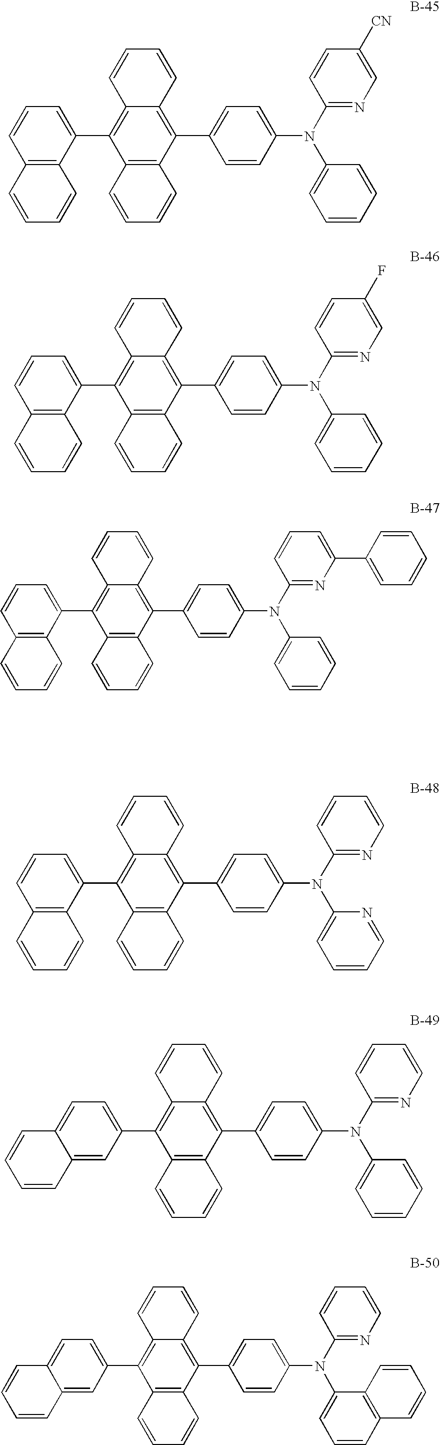

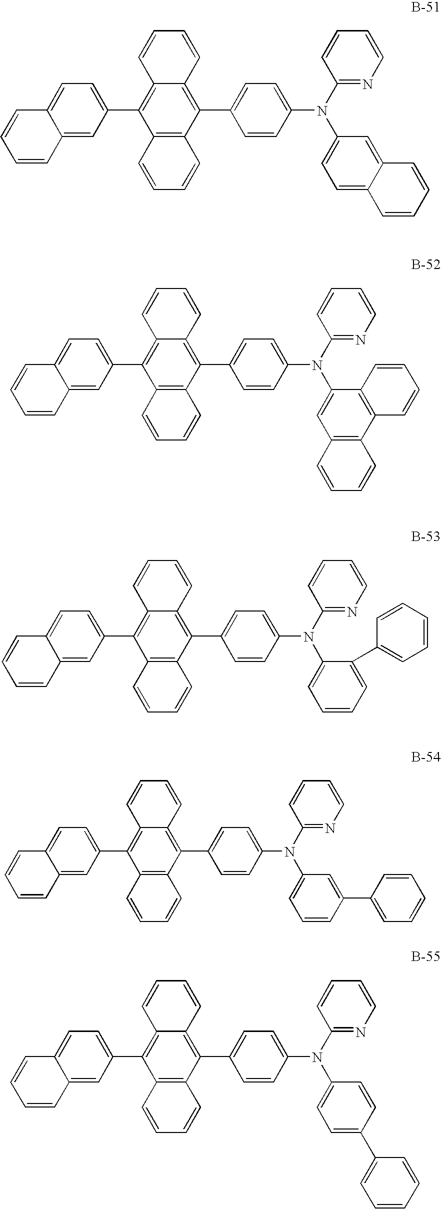

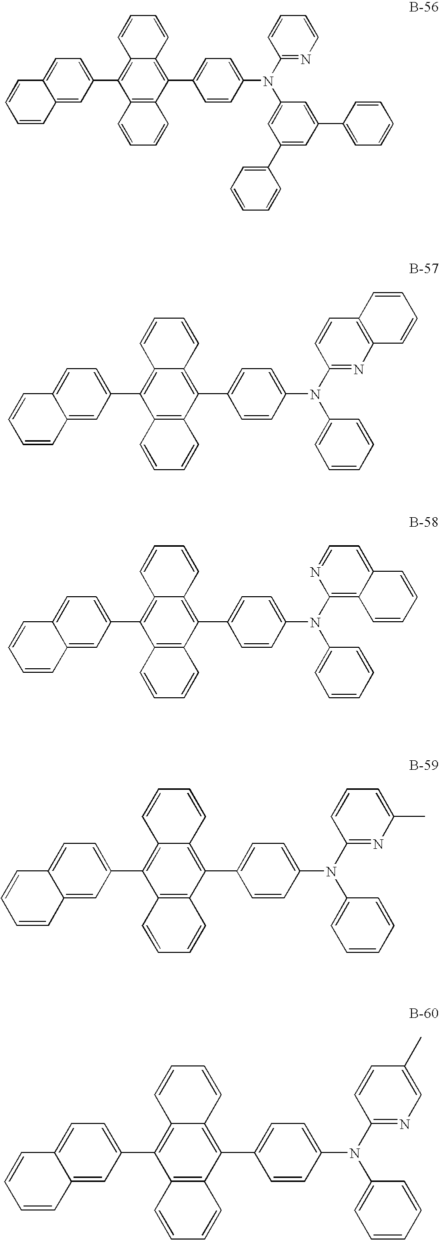

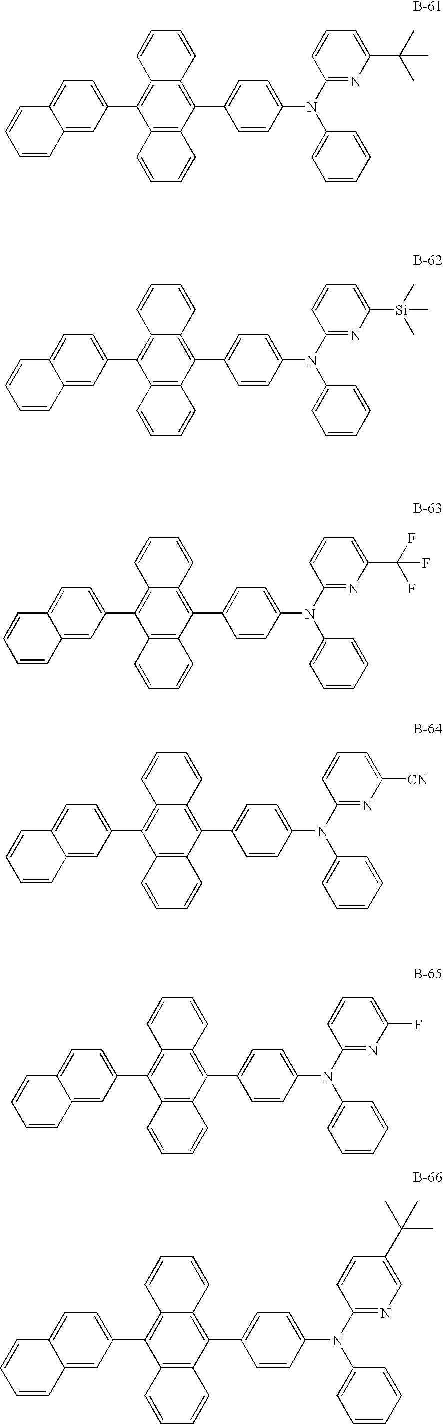

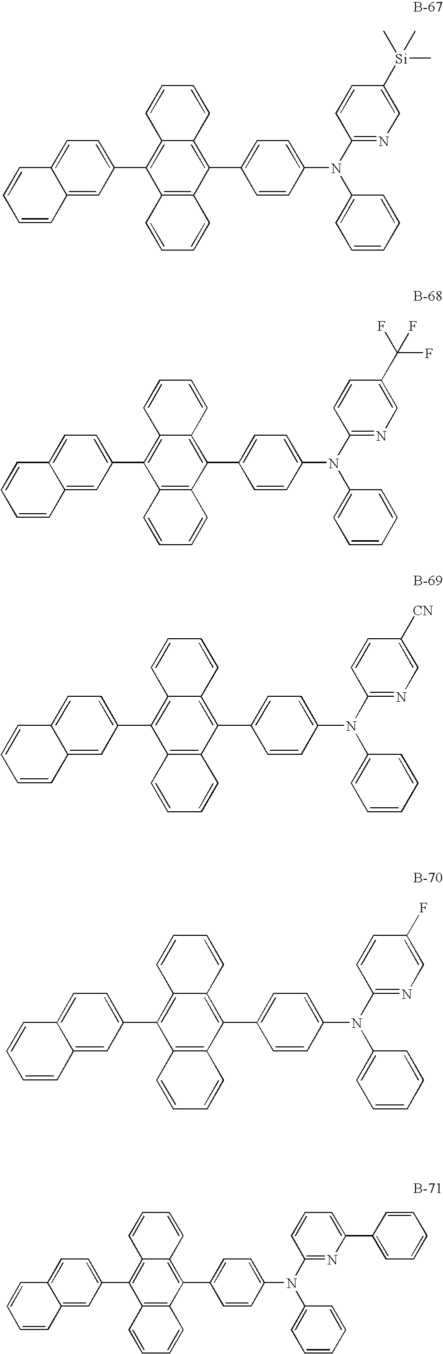

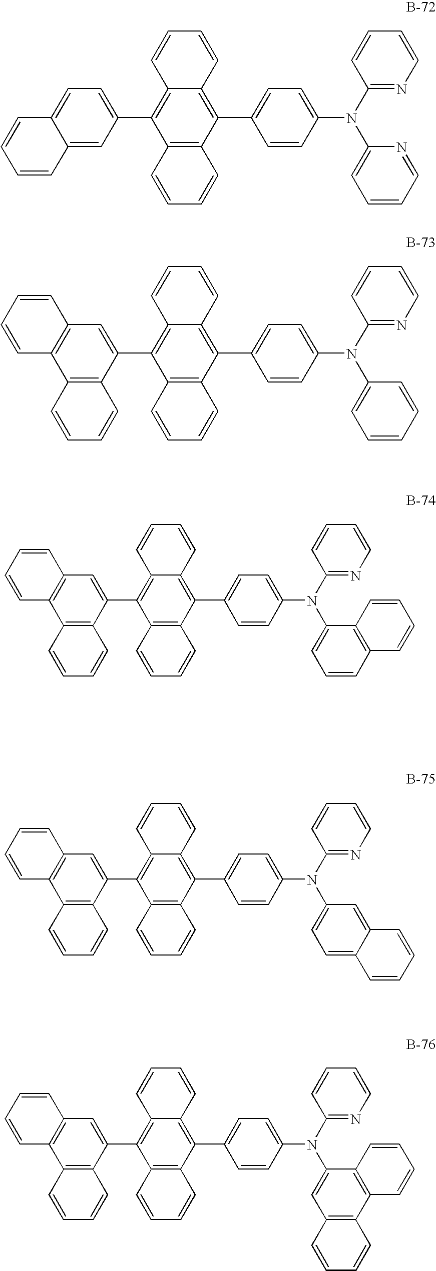

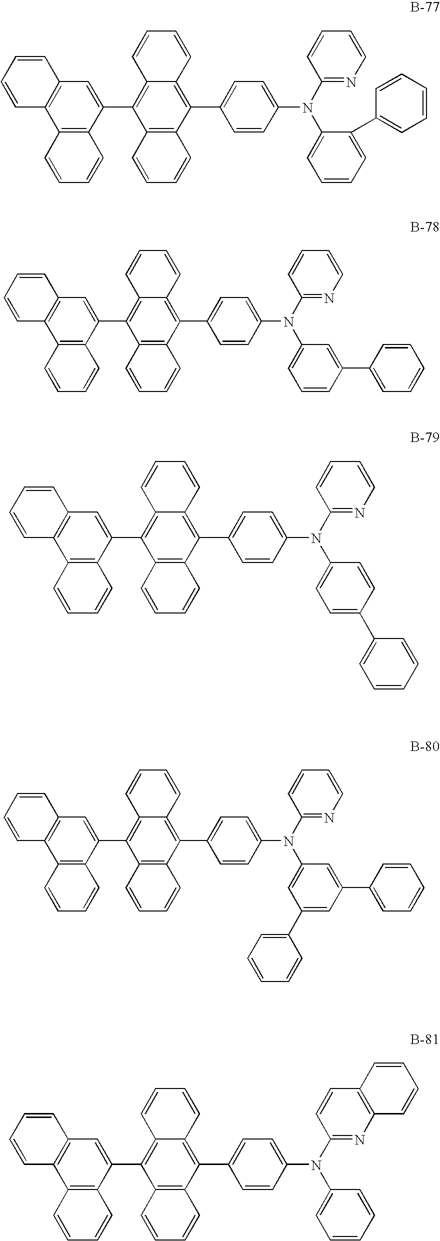

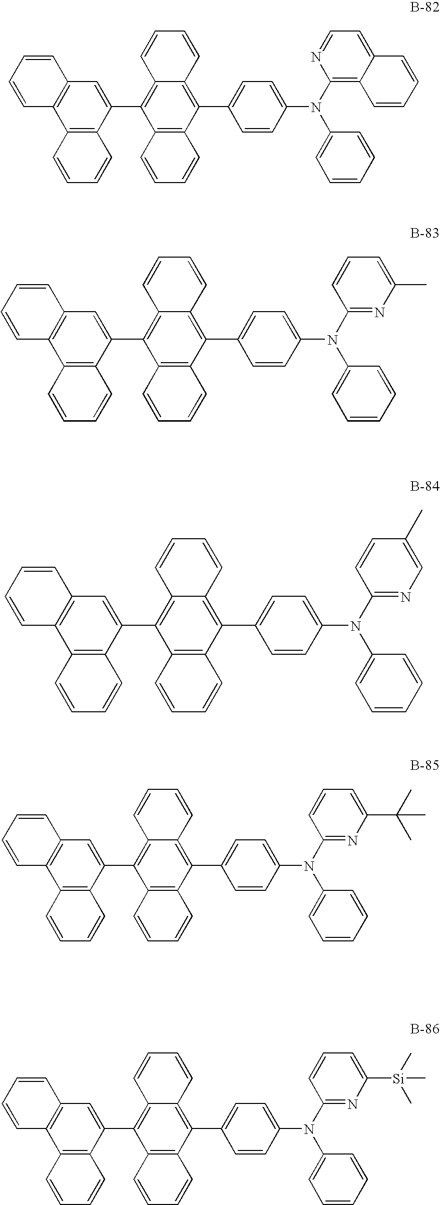

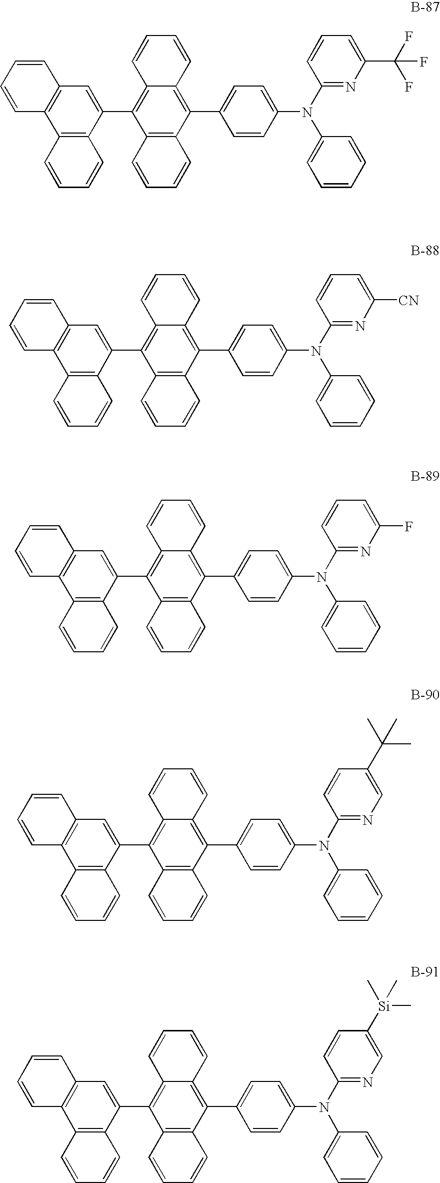

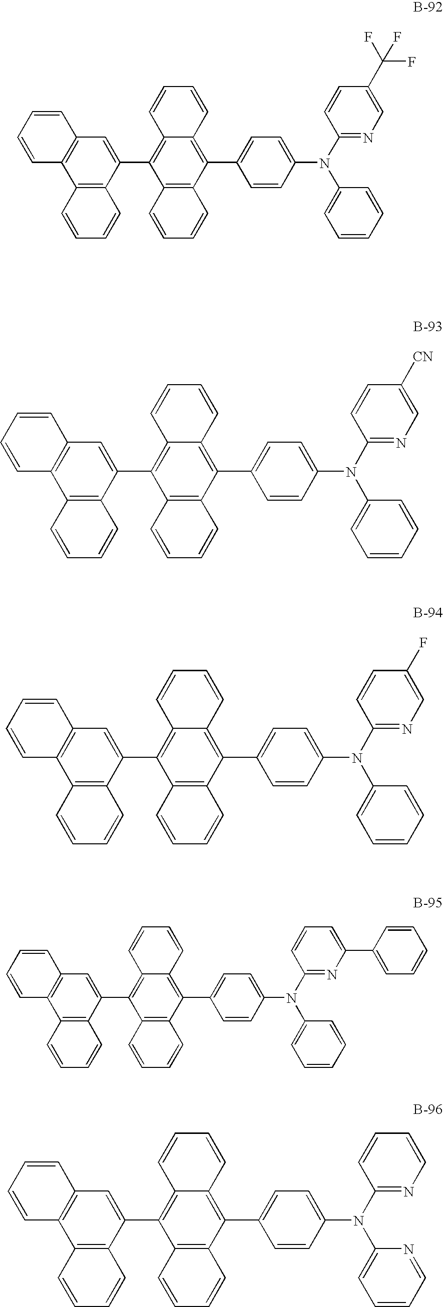

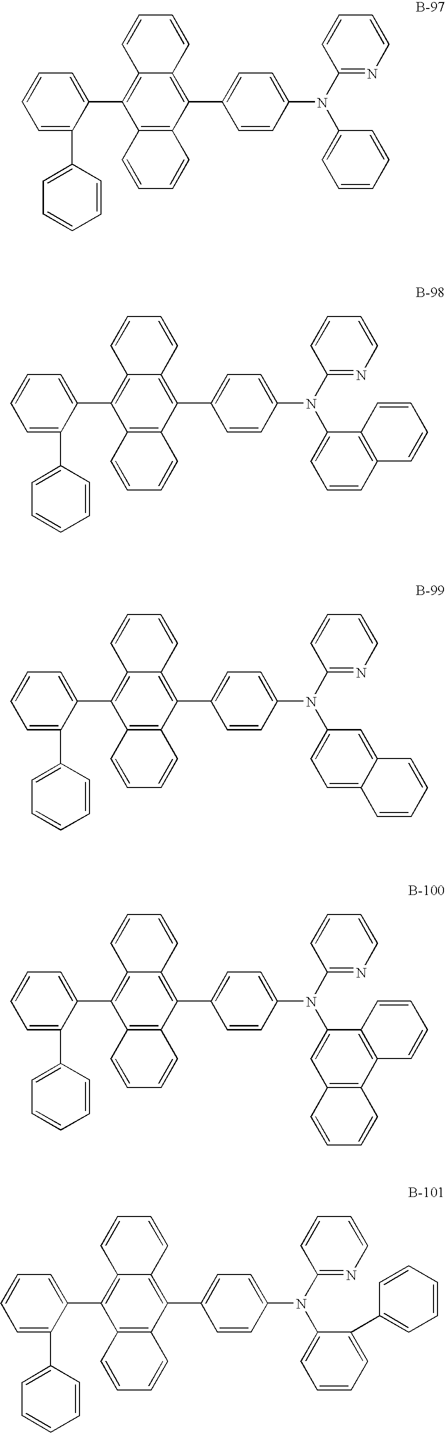

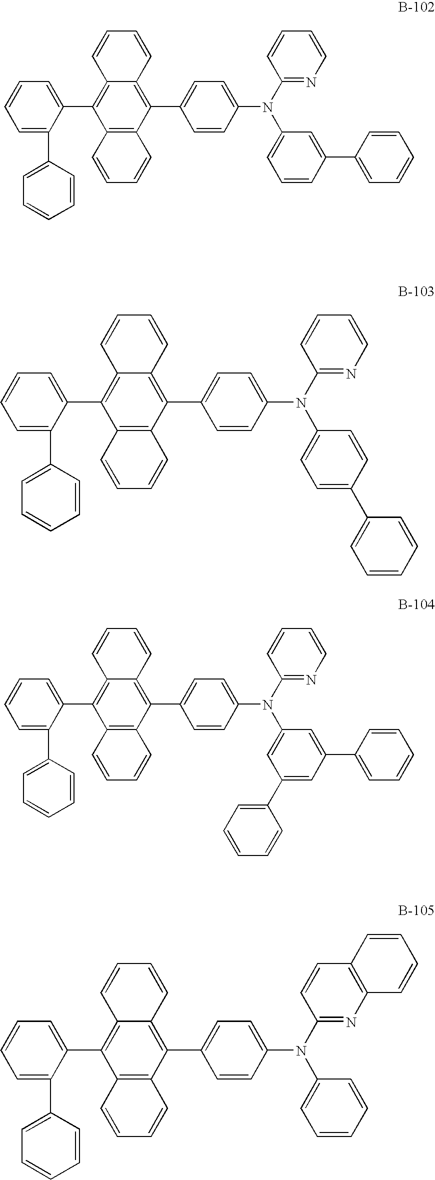

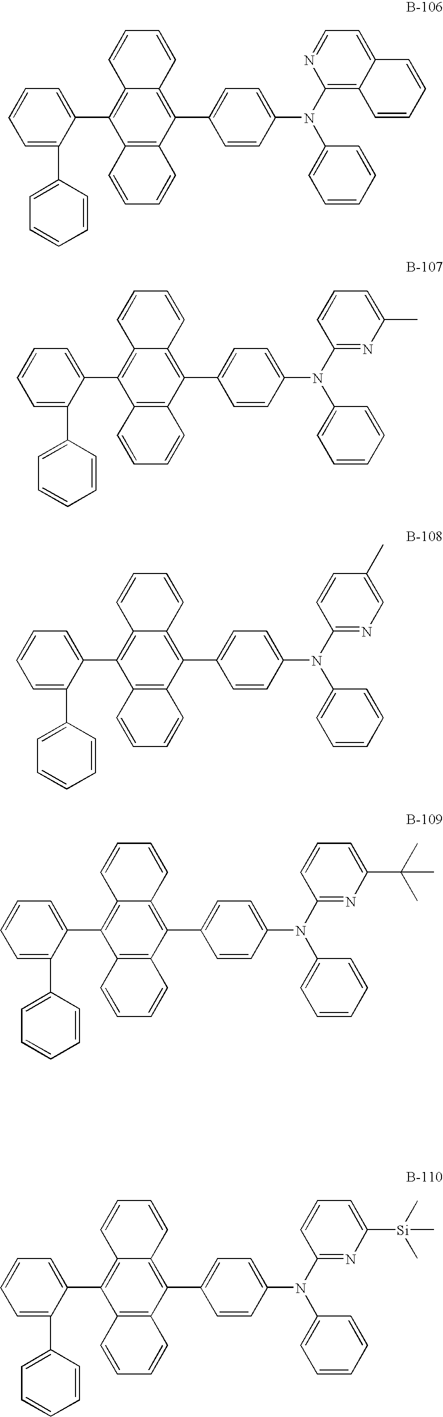

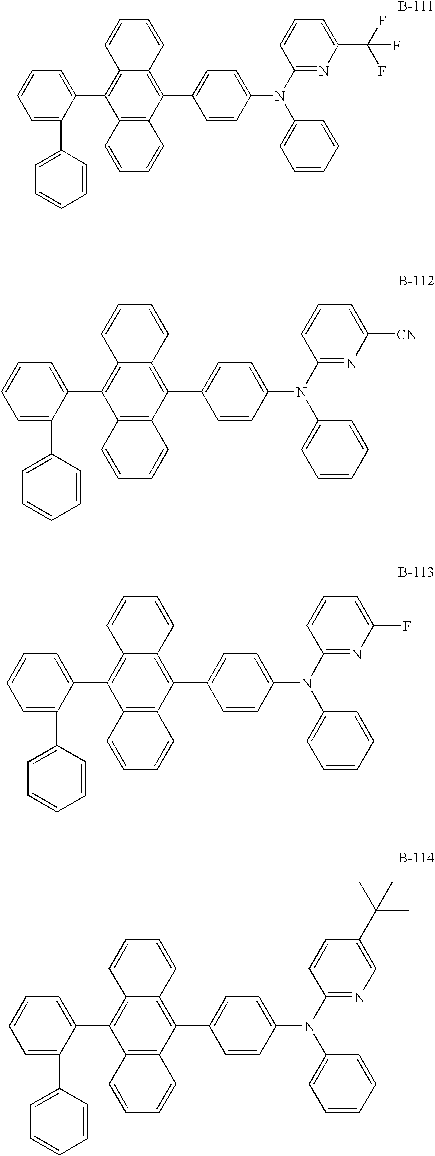

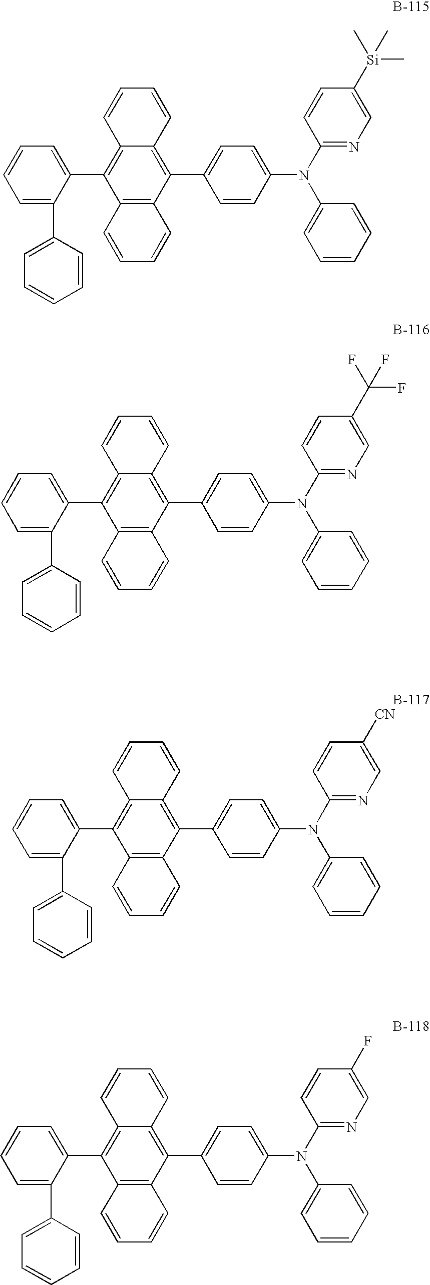

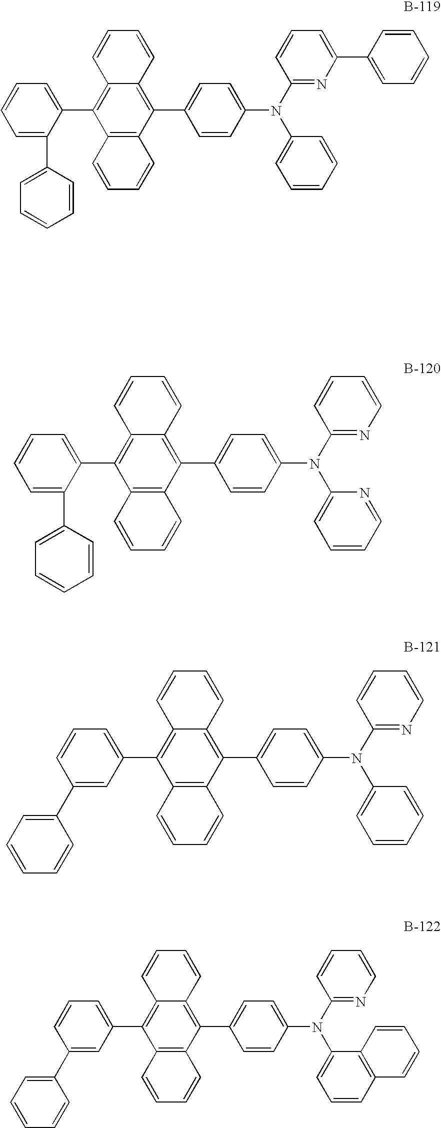

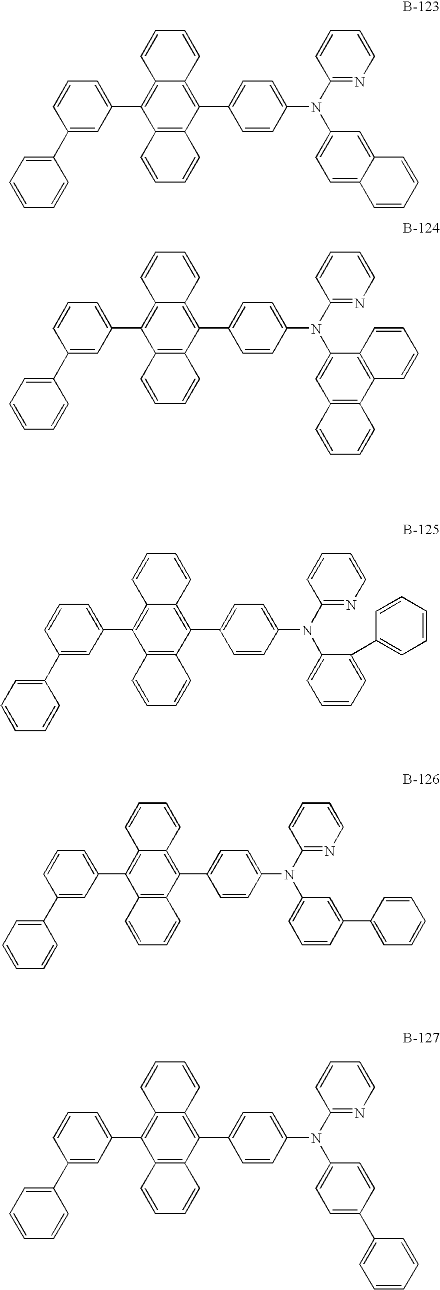

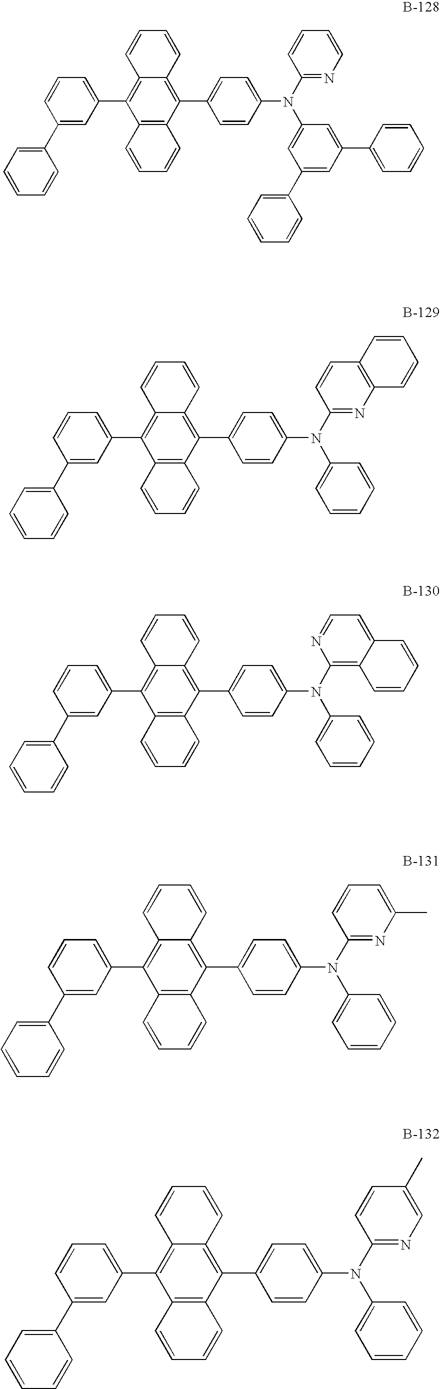

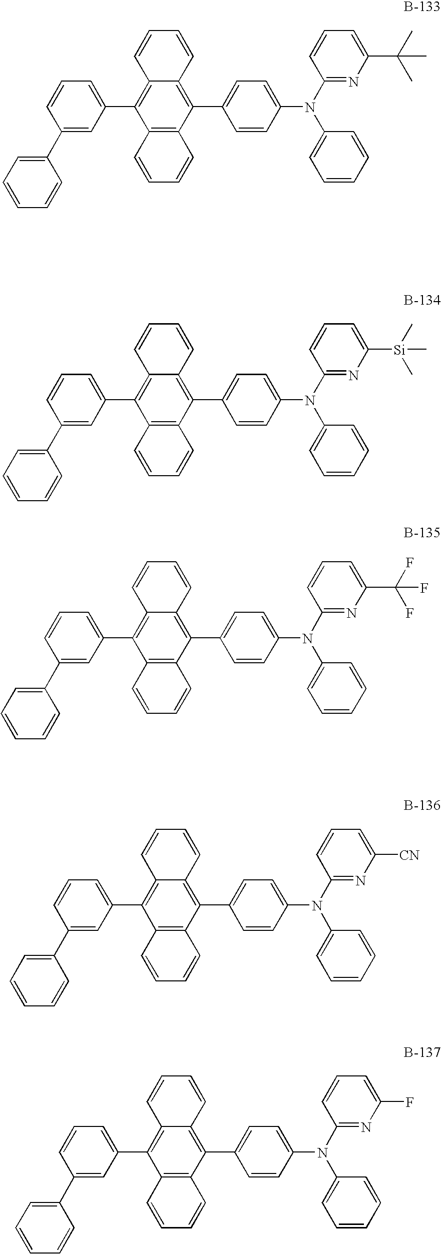

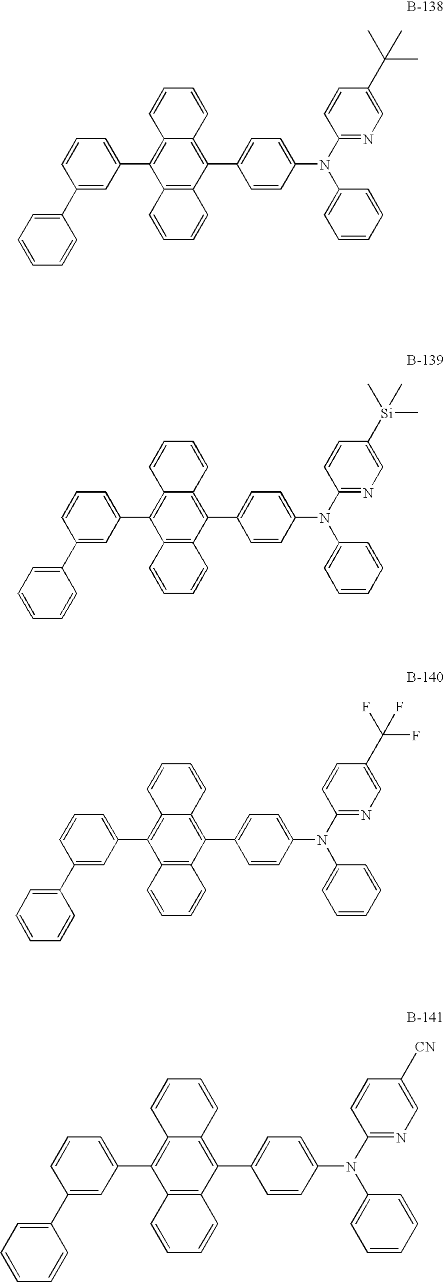

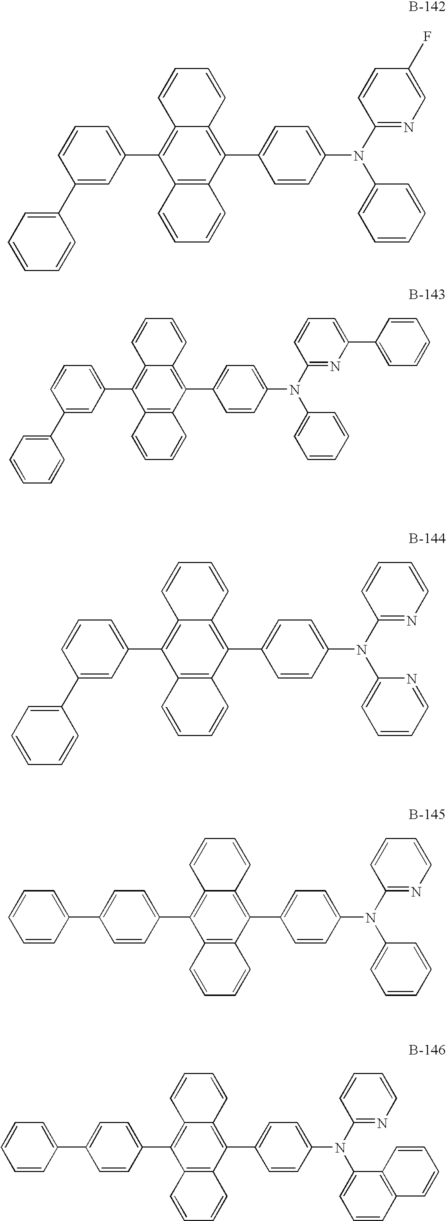

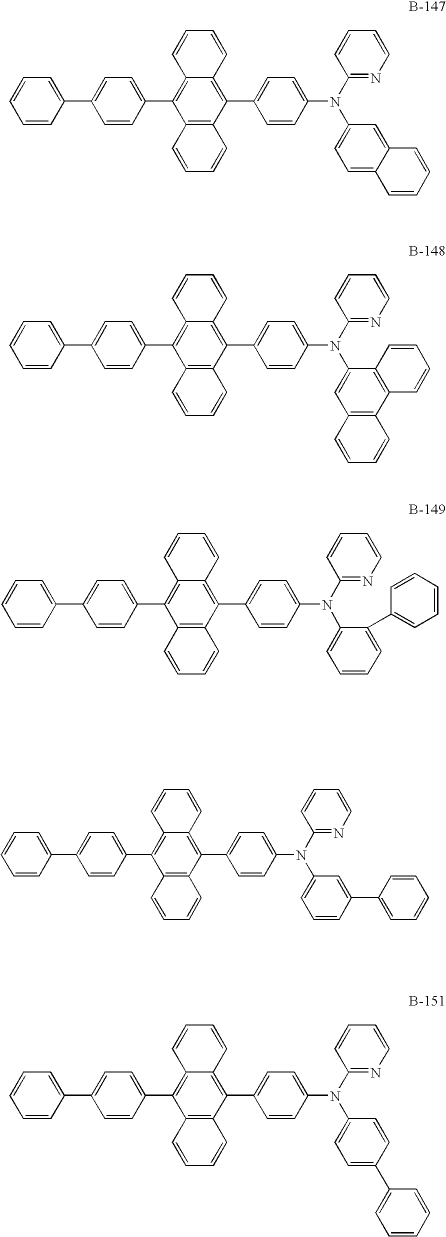

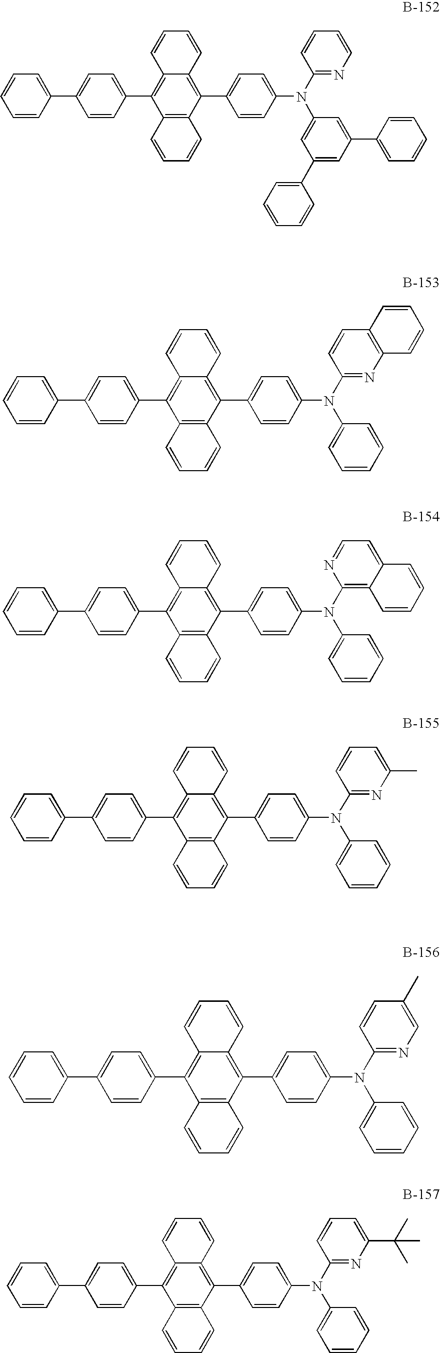

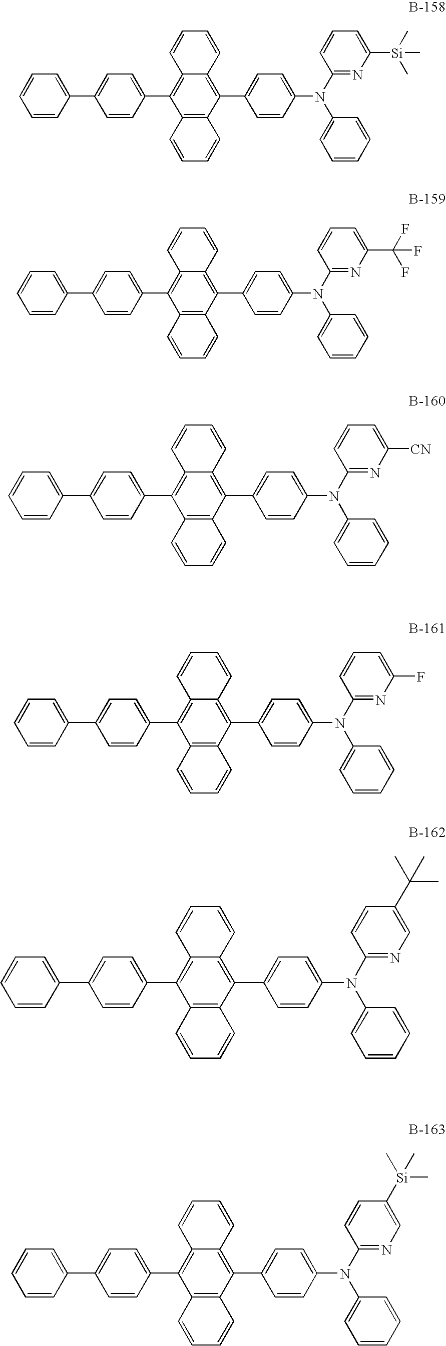

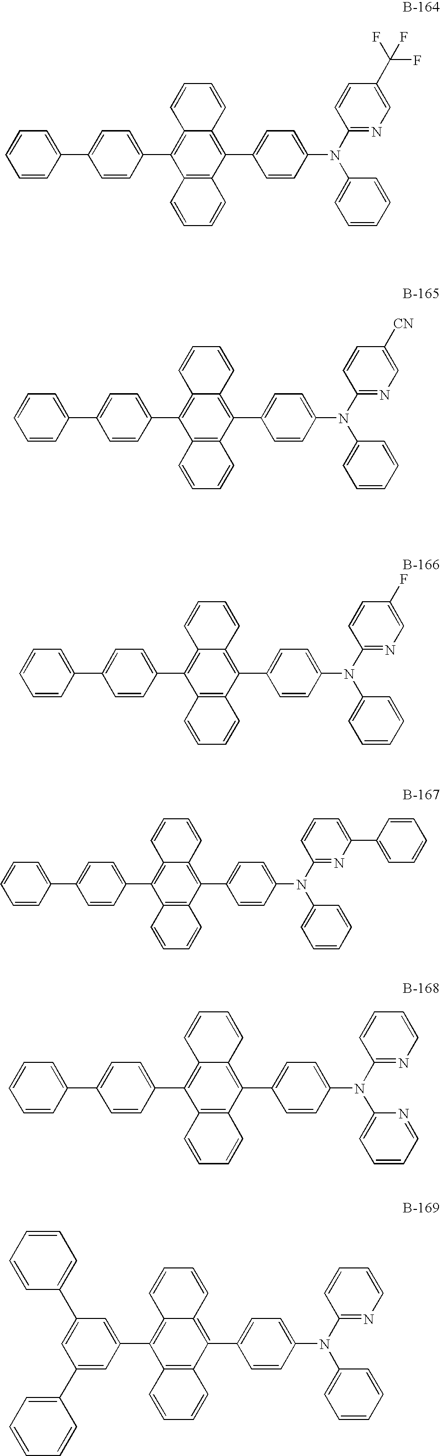

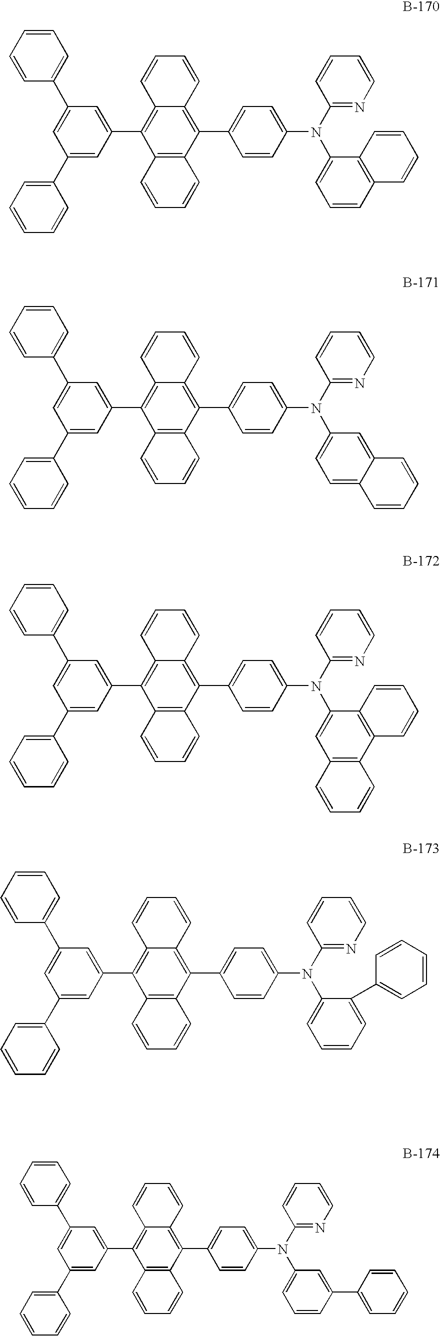

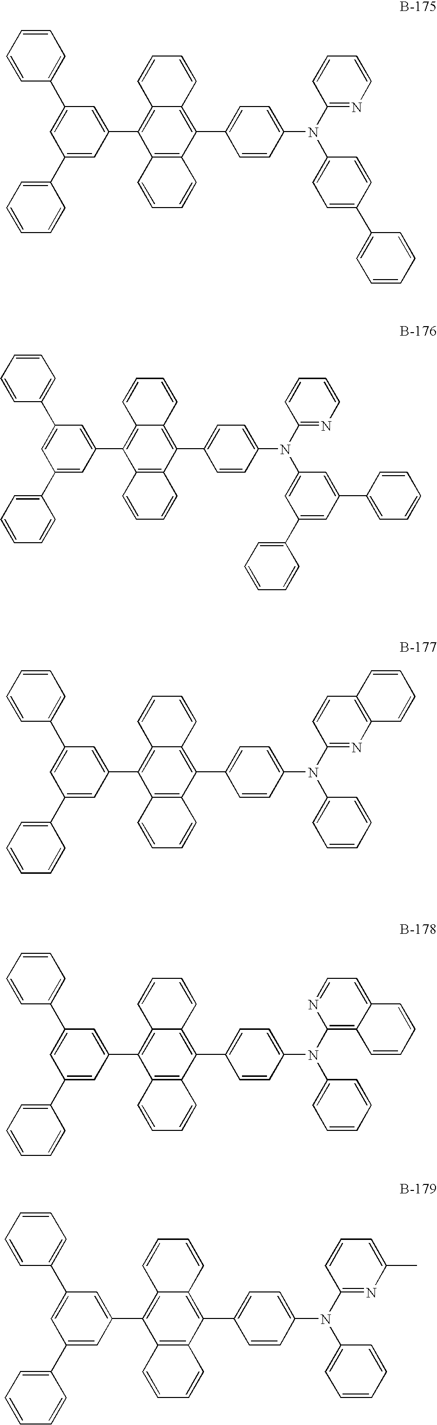

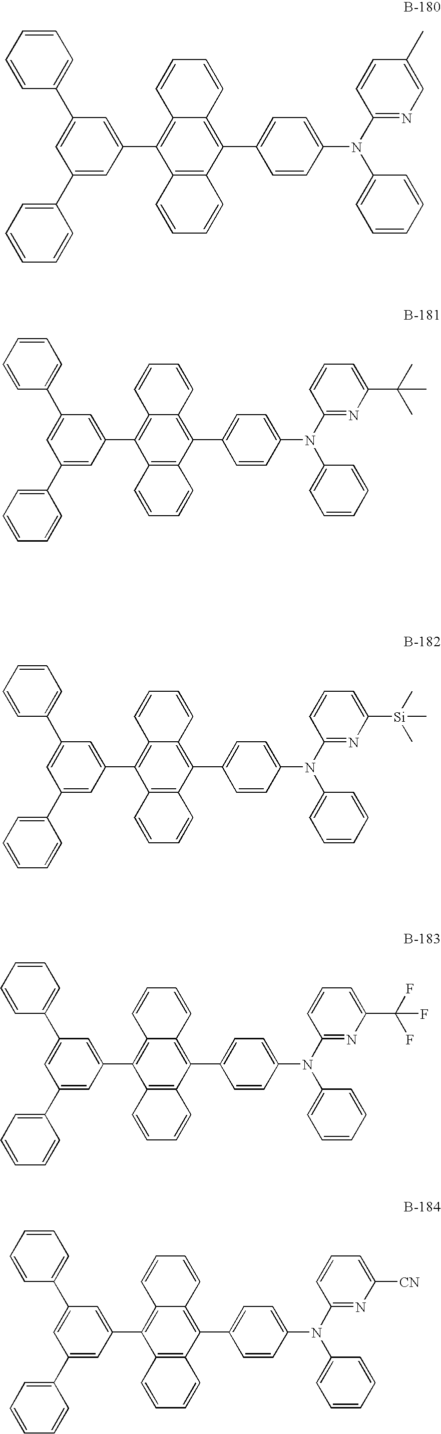

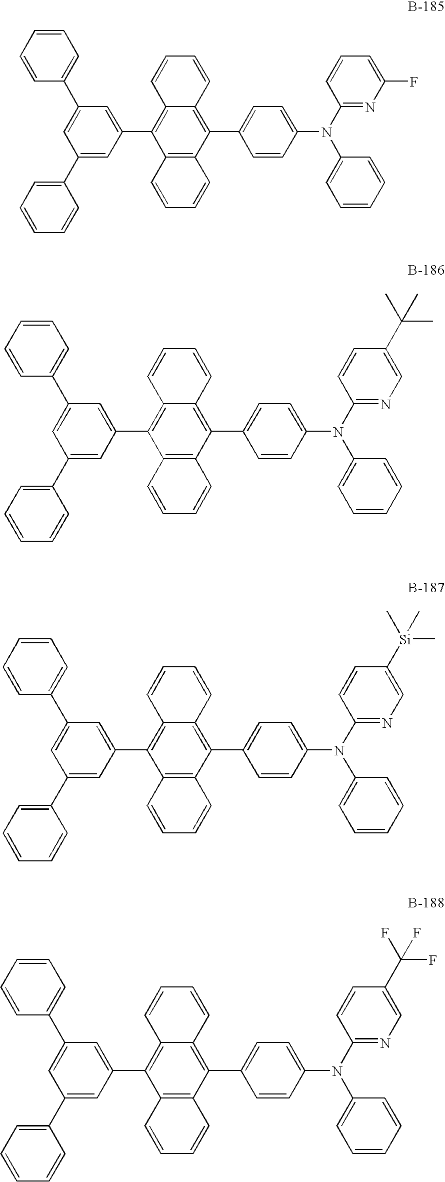

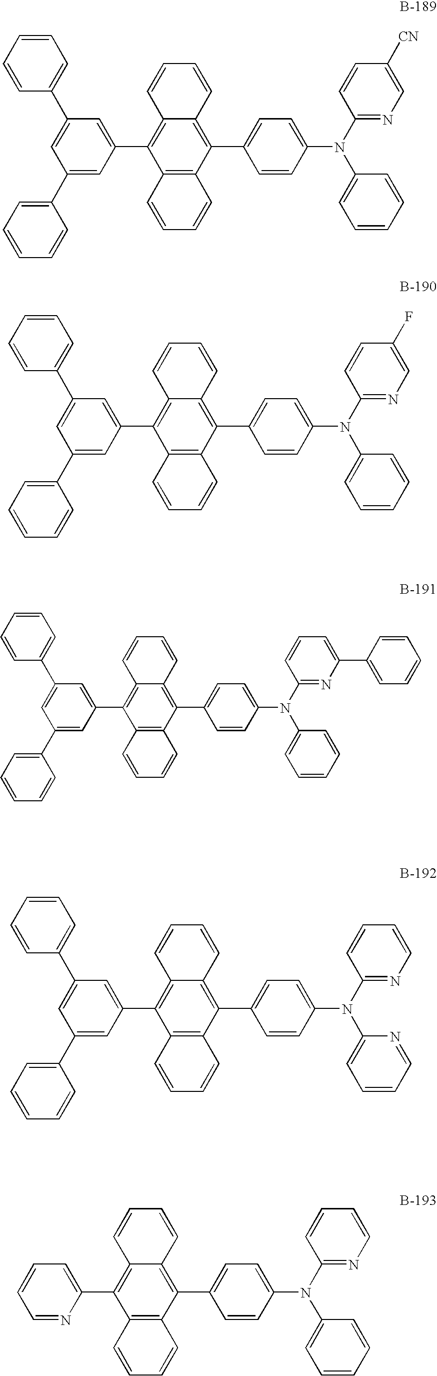

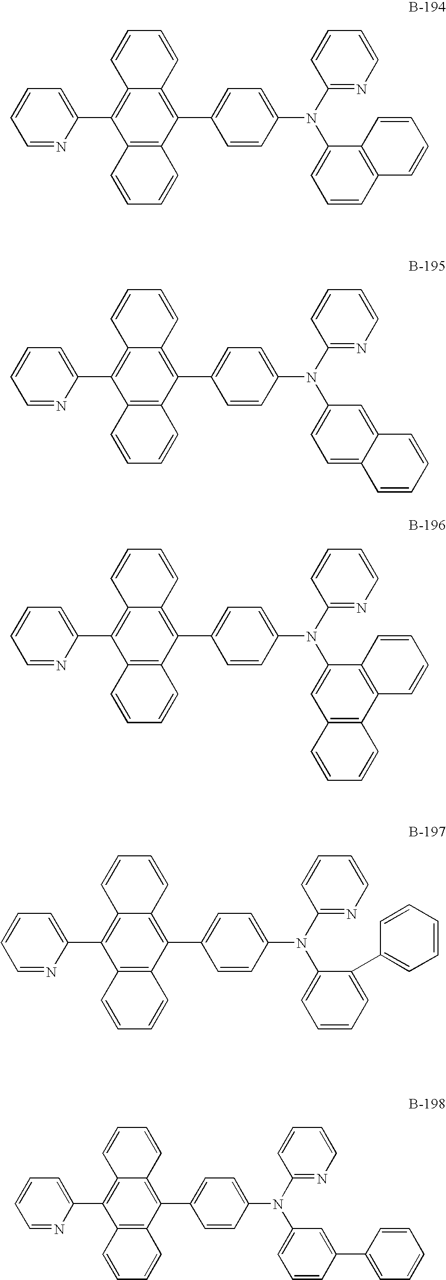

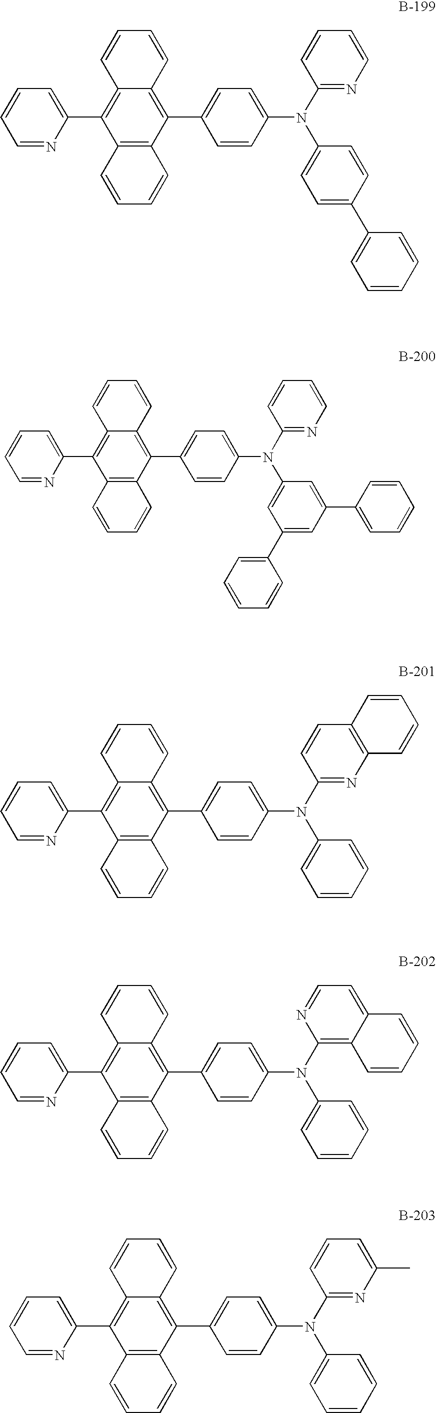

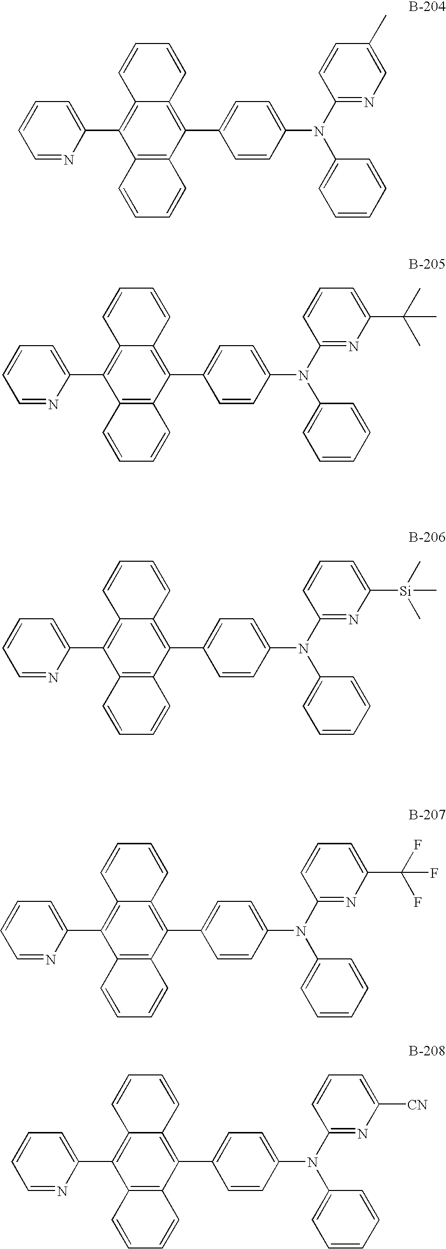

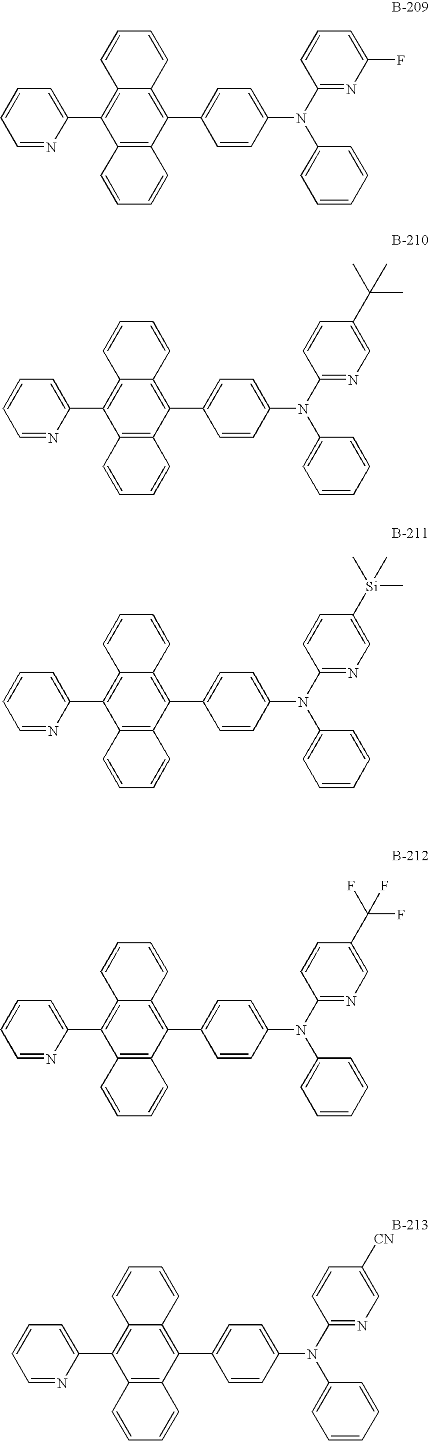

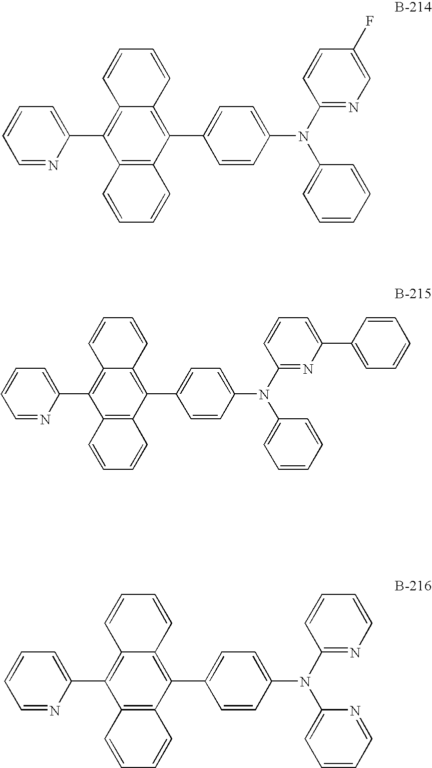

### Synthesis

A synthesis example of the electron transporting-injection compound marked by B-25 in the above Formula 5 is explained. The B-25 electron transporting-injection compound is 9-(1-naphthyl)-10-phenyl-(phenyl-2-pyridyl) anthracene.

### 1. Synthesis of phenyl-2-pyridylamine

phenyl-2-pyridylamine is synthesized by following Reaction Formula 4.

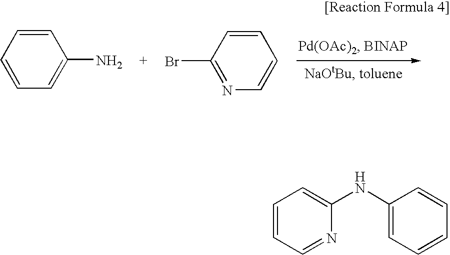

Aniline (10 g, 0.1 mol), 2-bromopyridine (17 g, 0.1 mol), palladium acetate (0.08 g, 0.32 mmol), BINAP (2,2′-bis(diphenylphosphino)-1,1′-binaphthyl) (0.26 g, 0.42 mmol), and NaOtBu (15.2 g, 0.16 mol) are put in a two-neck round-bottom flask and dissolved in toluene (100 mL). Subsequently, the resulting solution is refluxed for 12 hours. After completion of the reaction, the solution is cooled to a room temperature, and toluene is evaporated. Methanol (30 mL) is added thereto, and the resulting residence is filtered. Next, by re-crystallizing and filtering with methylene chloride and methanol, phenyl-2-pyridylamine (12.6 g, yield: 70%) is yield.

### 2. Synthesis of 4-bromophenyl-(phenyl-2-pyridyl)amine

4-bromophenyl-(phenyl-2-pyridyl)amine is synthesized by following Reaction Formula 5.

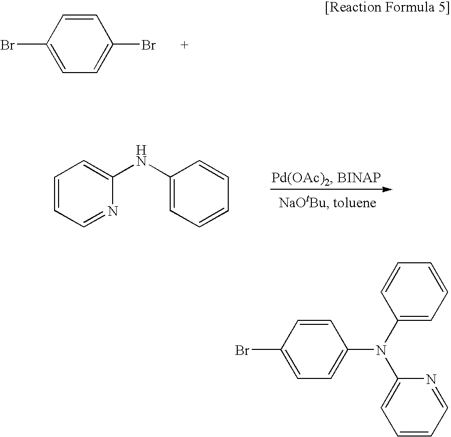

1,4-dibromobenzene (10 g, 0.04 mol), phenyl-2-pyridylamine (7.2 g, 0.04 mol), palladium acetate (0.18 g, 0.8 mmol), BINAP (0.7 g, 1.2 mmol) and NaOtBu (1.2 g, 0.13 mol) are put in a two-neck round-bottom flask and dissolved in toluene (80 mL). Subsequently, the resulting solution is refluxed for 12 hours. After completion of the reaction, the solution is cooled to a room temperature, and toluene is evaporated. Methanol (20 mL) is added thereto, and the resulting residence is filtered. Next, by re-crystallizing and filtering with methylene chloride and methanol, 4-bromophenyl-(phenyl-2-pyridyl)amine (9.6 g, yield: 70%) is yield.

### 3. Synthesis of 9-bromo-10-(1-naphthyl)anthracene

9-bromo-10-(1-naphthyl)anthracene is synthesized by following Reaction Formula 6.

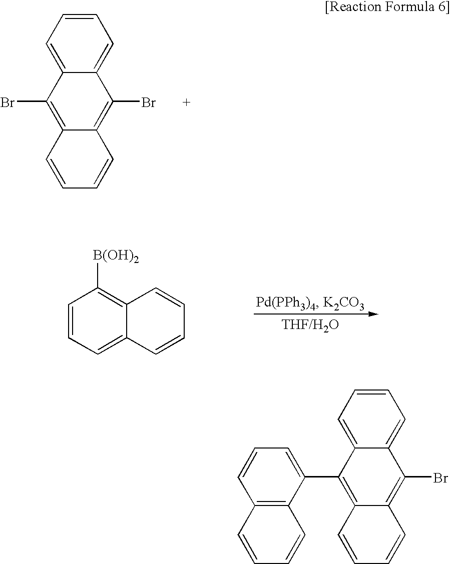

9,10-dibromoanthracene (5.0 g, 14.9 mol), 1-naphthyl-boronic acid (2.6 g, 14.9 mmol), tetrakis(triphenylphosphine)palladium(0) (Pd(PPh₃)₄ (0.5 g, 0.4 mmol), and a solution (100 mL) of 2M-K₂CO₃ and tetrahydrofuran (THF), where a ratio of 2M-K₂CO₃ to THF is 1:1, are put in a two-neck round-bottom flask and refluxed for 12 hours. After completion of the reaction, the resulting solution is cooled to a room temperature and extracted by using methylene chloride. The solvent is evaporated, and then being refined through a silica gel column to yield 9-bromo-10-(1-naphthyl)anthracene (4.0 g, yield: 70%).

### 4. Synthesis of 9-(1-naphthyl)-10-anthracenceboronic acid

9-(1-naphthyl)-10-anthracenceboronic acid is synthesized by following Reaction Formula 7.

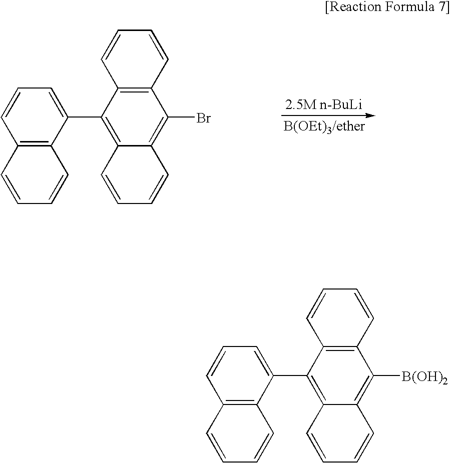

9-bromo-10-(1-naphthyl)anthracene (4.0 g, 0.01 mol) and ether (80 mL) are put in a two-neck round-bottom flask and stirred. The resulting solution is cooled into −78° C. using a dry-ice bath, 2.5M n-BuLi (4.6 mL, 0.01 mol) is dropped thereto, and then being stirred under a room temperature for 1 hour. After cooled again into −78° C. using a dry-ice bath, triethylborate (2.3 g, 0.017 mol) is dropped thereto, and then being stirred under a room temperature for 4 hours. Next, 2N HCL (100 mL) is put to the solution, and then being quenched. The solvent is evaporated, and the resulted solid is filtered. The solid is cleaned three or four times with a distilled water and hexane to yield 9-(1-naphthyl)-10-anthracenceboronic acid (2.5 g, yield: 70%).

### 5. Synthesis of 9-(1-naphthyl)-10-phenyl-(phenyl-2-pyridyl)anthracene

9-(1-naphthyl)-10-phenyl-(phenyl-2-pyridyl)anthracene is synthesized by following Reaction Formula 8.

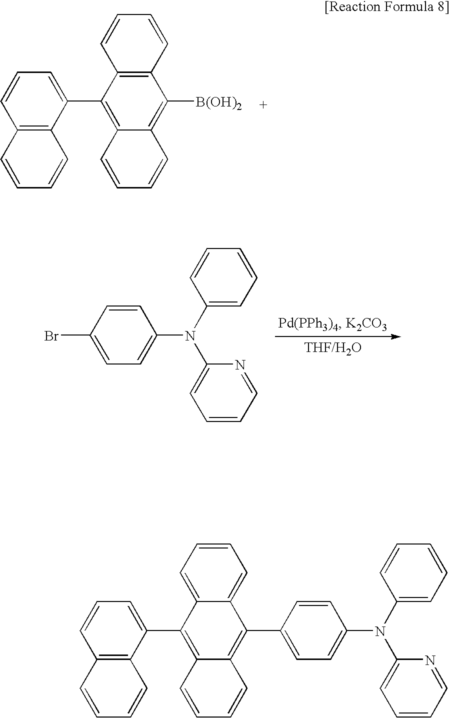

9-(1-naphthyl)-10-anthracenceboronic acid (2.0 g, 5.7 mmol), 4-bromophenyl(phenyl-2-pyridyl)amine (1.9 g, 5.7 mmol), Pd(PPh₃)₄ (0.2 g, 0.17 mmol), and a solution (100 mL) of 2M-K₂CO₃ and tetrahydrofuran (THF), where a ratio of 2M-K₂CO₃ to THF is 1:1, are put in a two-neck round-bottom flask and refluxed for 12 hours. The resulting solution is cooled into a room temperature, and then being extracted with methylene chloride. The solvent is evaporated, and then being refined through a silica gel column to yield 9-(1-naphthyl)-10-phenyl-(phenyl-2-pyridyl)anthracene (1.9 g, yield: 60%).

Hereinafter, a detailed description will be made of preferred examples associated with the OELD according to the present invention. More specifically, the examples relate to an OELD including the electron transporting-injection compound of Formula 4 as an electron transporting-injection layer. In following Examples 5 to 8, lithium fluoride (LiF) is used as an electron injection layer. Alternatively, the electron transporting-injection compound according to the present invention can be used as both an electron injection layer and an electron transporting layer without the LiF layer.

### EXAMPLES

### Example 5

An indium-tin-oxide (ITO) layer is patterned on a substrate and washed such that an emission area of the ITO layer is 3 mm*3 mm. The substrate is loaded in a vacuum chamber, and the process pressure is adjusted to 1*10⁻⁶ torn CuPC (about 650 angstroms) represented by the above Formula 3-1, 4,4′-bis[N-(1-naphtyl)-N-phenylamino]-biphenyl (NPD) (about 400 angstroms) represented by the above Formula 3-2, an emitting layer (about 200 angstroms) including DPVBi, which is represented by the above Formula 3-3, as a host and a compound, which is represented by the above Formula 3-4, as a dopant (about 1 weight %), an electron transporting-injection compound represented by B-01 in the above Formula 5 (about 350 angstroms), lithium fluoride (LiF) (about 5 angstroms) and aluminum (Al) (about 1000 angstroms) are sequentially formed on the ITO layer such that an OELD is fabricated.

The OELD produces a brightness of 730 cd/m² at an electric current of 0.9 mA and a voltage of 5.6 V. At this time, the X index and Y index of CIE color coordinates are 0.136 and 0.190, respectively.

### Example 6

An indium-tin-oxide (ITO) layer is patterned on a substrate and washed such that an emission area of the ITO layer is 3 mm*3 mm. The substrate is loaded in a vacuum chamber, and the process pressure is adjusted to 1*10⁻⁶ torn CuPC (about 650 angstroms) represented by the above Formula 3-1, 4,4′-bis[N-(1-naphtyl)-N-phenylamino]-biphenyl (NPD) (about 400 angstroms) represented by the above Formula 3-2, an emitting layer (about 200 angstroms) including DPVBi, which is represented by the above Formula 3-3, as a host and a compound, which is represented by the above Formula 3-4, as a dopant (about 1 weight %), an electron transporting-injection compound represented by B-12 in the above Formula 5 (about 350 angstroms), lithium fluoride (LiF) (about 5 angstroms) and aluminum (Al) (about 1000 angstroms) are sequentially formed on the ITO layer such that an OELD is fabricated.

The OELD produces a brightness of 690 cd/m² at an electric current of 0.9 mA and a voltage of 5.8 V. At this time, the X index and Y index of CIE color coordinates are 0.138 and 0.200, respectively.

### Example 7

An indium-tin-oxide (ITO) layer is patterned on a substrate and washed such that an emission area of the ITO layer is 3 mm*3 mm. The substrate is loaded in a vacuum chamber, and the process pressure is adjusted to 1*10⁻⁶ torr. CuPC (about 650 angstroms) represented by the above Formula 3-1, 4,4′-bis[N-(1-naphtyl)-N-phenylamino]-biphenyl (NPD) (about 400 angstroms) represented by the above Formula 3-2, an emitting layer (about 200 angstroms) including DPVBi, which is represented by the above Formula 3-3, as a host and a compound, which is represented by the above Formula 3-4, as a dopant (about 1 weight %), an electron transporting-injection compound represented by B-13 in the above Formula 5 (about 350 angstroms), lithium fluoride (LiF) (about 5 angstroms) and aluminum (Al) (about 1000 angstroms) are sequentially formed on the ITO layer such that an OELD is fabricated.

The OELD produces a brightness of 710 cd/m² at an electric current of 0.9 mA and a voltage of 5.7 V. At this time, the X index and Y index of CIE color coordinates are 0.136 and 0.189, respectively.

### Example 8

An indium-tin-oxide (ITO) layer is patterned on a substrate and washed such that an emission area of the ITO layer is 3 mm*3 mm. The substrate is loaded in a vacuum chamber, and the process pressure is adjusted to 1*10⁻⁶ torn CuPC (about 650 angstroms) represented by the above Formula 3-1, 4,4′-bis[N-(1-naphtyl)-N-phenylamino]-biphenyl (NPD) (about 400 angstroms) represented by the above Formula 3-2, an emitting layer (about 200 angstroms) including DPVBi, which is represented by the above Formula 3-3, as a host and a compound, which is represented by the above Formula 3-4, as a dopant (about 1 weight %), an electron transporting-injection compound represented by B-14 in the above Formula 5 (about 350 angstroms), lithium fluoride (LiF) (about 5 angstroms) and aluminum (Al) (about 1000 angstroms) are sequentially formed on the ITO layer such that an OELD is fabricated.

The OELD produces a brightness of 706 cd/m² at an electric current of 0.9 mA and a voltage of 5.7 V. At this time, the X index and Y index of CIE color coordinates are 0.137 and 0.192, respectively.

### Comparative Example 2

An indium-tin-oxide (ITO) layer is patterned on a substrate and washed such that an emission area of the ITO layer is 3 mm*3 mm. The substrate is loaded in a vacuum chamber, and the process pressure is adjusted to 1*10⁻⁶ torr. CuPC (about 650 angstroms) represented by the above Formula 3-1, 4,4′-bis[N-(1-naphtyl)-N-phenylamino]-biphenyl (NPD) (about 400 angstroms) represented by the above Formula 3-2, an emitting layer (about 200 angstroms) including DPVBi, which is represented by the above Formula 3-3, as a host and a compound, which is represented by the above Formula 3-4, as a dopant (about 1 weight %), Alq3 (about 350 angstroms) represented by the above Formula 3-5, lithium fluoride (LiF) (about 5 angstroms) and aluminum (Al) (about 1000 angstroms) are sequentially formed on the ITO layer such that an OELD is fabricated.

The OELD produces a brightness of 655 cd/m² at an electric current of 0.9 mA and a voltage of 6.4 V. At this time, the X index and Y index of CIE color coordinates are 0.136 and 0.188, respectively.

The OELD fabricated in Examples 5 to 8 and Comparative Example 2 is evaluated for efficiency, brightness, and so on. A voltage has a dimension of [V], an electric current has a dimension of [mA], a brightness has a dimension of [cd/m²], a current efficiency has a dimension of [cd/A], and a power efficiency has a dimension of [1 m/W]. The evaluated results are shown in Table 2.

TABLE 2

| | | Electric | Bright- | Current | Power | CIE | CIE |
|---|---|---|---|---|---|---|---|
| | voltage | current | ness | efficiency | efficiency | (X) | (Y) |
| Ex. 5 | 5.6 | 0.9 | 721 | 7.2 | 4.03 | 0.136 | 0.190 |
| Ex. 6 | 5.8 | 0.9 | 690 | 6.9 | 3.73 | 0.138 | 0.200 |
| Ex. 7 | 5.7 | 0.9 | 710 | 7.1 | 3.91 | 0.136 | 0.189 |
| Ex. 8 | 5.7 | 0.9 | 706 | 7.0 | 3.86 | 0.137 | 0.192 |
| Com. | 6.7 | 0.9 | 526 | 5.26 | 2.47 | 0.136 | 0.188 |
| Ex. 2 | | | | | | | |

As shown in Table 2, the OELD in Examples 5 to 8 has improved luminescent efficiency such that power consumption for the OELD is reduced. As a result, since the OELD using the electron transporting-injection compound can be driven a low driving voltage, power consumption is reduced and a lifetime of the OELD using the electron transporting-injection compound according to the present invention is improved.

FIG. 2 is a schematic cross-sectional view of an OELD according to the present invention. In FIG. 2, an OELD includes a first substrate (not shown), a second substrate (not shown) facing the first substrate 101, and an organic electroluminescent diode E between the first and second substrates.

The organic electroluminescent diode E includes a first electrode 110 as an anode, a second electrode 130 as a cathode, and an organic emitting layer 120 between the first and second electrodes 110 and 130.

The first electrode 110 is formed of a material having a large work function. For example, the first electrode 110 may be formed of ITO. The second electrode 130 is formed of a material having a small work function. For example, the second electrode 130 may be formed of one of Al and Al alloy (AlNd).

The organic emitting layer 120 has red, green and blue organic emitting patterns. To maximize luminescent efficiency, the organic emitting layer 120 includes a hole injection layer (HIL) 122 on the first electrode 110, a hole transporting layer (HTL) 124 on the HIL 122, an emitting material layer (EML) 126 on the HTL 124, and an electron transporting-injection layer 128 on the EML 126 and under the second electrode 130. The electron transporting-injection layer 128 is formed of one of electron transporting-injection compounds in the above Formulas 2 and 5. The organic emitting layer 120 may further include an electron injection layer (not shown) between the electron transporting-injection layer 128 and the second electrode 130. For example, the electron injection layer 122 may be formed of CuPC, and the electron transporting layer 124 may be formed of NPD. The electron injection layer (not shown) may be formed of LiF.

The OELD using the electron transporting-injection compound can be driven a low driving voltage, power consumption is reduced and a lifetime of the OELD using the electron transporting-injection compound according to the present invention is improved.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

What is claimed is:

1. An electron transporting-injection compound, represented by following Formula 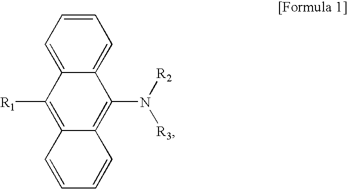
wherein each of the R1, the R2 and the R3 is selected from substituted or non-substituted aromatic group, substituted or non-substituted heterocyclic group, or of substituted or non-substituted aliphatic group, and at least one of the R2 and the R3 is selected from substituted or non-substituted heterocyclic group.

2. The compound according to claim 1, wherein the substituted or non-substituted heterocyclic group for the at least one of R2 and R3 is pyridyl such that the compound of the Formula 1 is represented by following Formula 2.

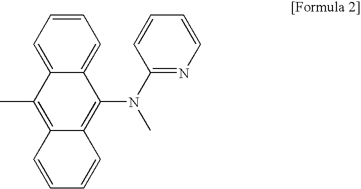

3. The compound according to claim 1, wherein the aromatic group includes phenyl, byphenyl, naphthyl, phenanthrenyl, and terphenyl, and the heterocyclic group includes pyridyl, bipyridyl, phenylpyridyl, pyridylphenyl, terpyridyl, quinolinyl, isoquinolinyl, and quinoxalinyl, and wherein the aliphatic group includes methyl, ethyl, propyl, isopropyl, butyl, and tert-butyl.

4. The compound according to claim 1, wherein a substituent for each of the R1, the R2 and the R3 is one of aryl, alkyl, alkoxy, allyamino, alkylamino, amino, halogen and cyano.

5. The compound according to claim 4, wherein the substituent for each of the R1, the R2 and the R3 is one of methyl, ethyl, propyl, isopropyl, tert-butyl, methoxy, ethoxy, butoxy, trimethylsilyl, fluorine and chlorine.

6. The compound according to claim 1, wherein when at least one of the R1, the R2 and R3 is 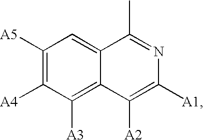
at least one of the A1 to the A5 is methyl.

7. The compound according to claim 1, wherein when at least one of the R1, the R2 and R3 is 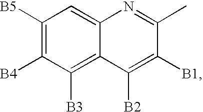
at least one of the B1 to the B5 is methyl.

8. An electron transporting-injection compound, represented by following Formula 1: 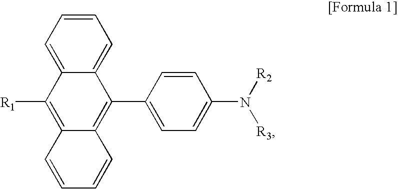
wherein each of the R1, the R2 and the R3 is selected from substituted or non-substituted aromatic group, substituted or non-substituted heterocyclic group, or of substituted or non-substituted aliphatic group, and at least one of the R2 and the R3 is selected from substituted or non-substituted heterocyclic group.

9. The compound according to claim 8, wherein the substituted or non-substituted heterocyclic group for the at least one of R2 and R3 is pyridyl such that the compound of the Formula 1 is represented by following Formula 2.

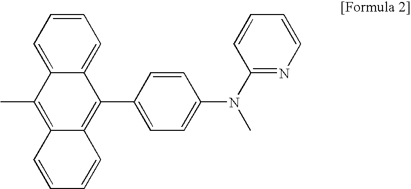

10. The compound according to claim 8, wherein the aromatic group includes phenyl, byphenyl, naphthyl, phenanthrenyl, and terphenyl, and the heterocyclic group includes pyridyl, bipyridyl, phenylpyridyl, pyridylphenyl, terpyridyl, quinolinyl, isoquinolinyl, and quinoxalinyl, and wherein the aliphatic group includes methyl, ethyl, propyl, isopropyl, butyl, and tert-butyl.

11. The compound according to claim 8, wherein a substituent for each of the R1, the R2 and the R3 is one of aryl, alkyl, alkoxy, allyamino, alkylamino, amino, halogen and cyano.

12. The compound according to claim 11, wherein the substituent for each of the R1, the R2 and the R3 is one of methyl, ethyl, propyl, isopropyl, tert-butyl, methoxy, ethoxy, butoxy, trimethylsilyl, fluorine and chlorine.

13. The compound according to claim 8, wherein when at least one of the R1, the R2 and R3 is 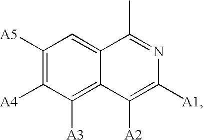
at least one of the A1 to the A5 is methyl.

14. The compound according to claim 8, wherein when at least one of the R1, the R2 and R3 is 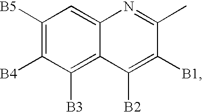
at least one of the B1 to the B5 is methyl.

15. An organic electroluminescent device, comprising:
a first electrode;
a second electrode facing the first electrode; and
an organic emitting layer positioned between the first and second electrodes and including a hole injection layer on the first electrode, a hole transporting layer on the hole injection layer, an emitting material layer on the transporting hole injection layer and an electron transporting-injection layer on the emitting material layer, wherein the electron transporting-injection layer formed of an electron transporting-injection compound represented by following Formula 1: 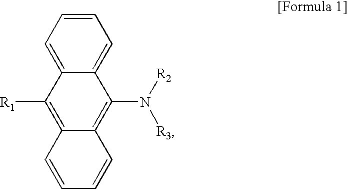
wherein each of the R1, the R2 and the R3 is selected from substituted or non-substituted aromatic group, substituted or non-substituted heterocyclic group, or of substituted or non-substituted aliphatic group, and at least one of the R2 and the R3 is selected from substituted or non-substituted heterocyclic group.

16. The device according to claim 15, wherein the organic emitting layer further includes an electron injection layer between the electron transporting-injection layer and the second electrode.

17. An organic electroluminescent device, comprising:
a first electrode;
a second electrode facing the first electrode; and
an organic emitting layer positioned between the first and second electrodes and including a hole injection layer on the first electrode, a hole transporting layer on the hole injection layer, an emitting material layer on the hole transporting injection layer and an electron transporting-injection layer on the emitting material layer, wherein the electron transporting-injection layer formed of an electron transporting-injection compound represented by following Formula 1: 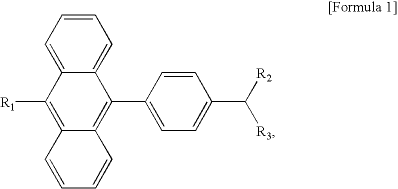
wherein each of the R1, the R2 and the R3 is selected from substituted or non-substituted aromatic group, substituted or non-substituted heterocyclic group, or of substituted or non-substituted aliphatic group, and at least one of the R2 and the R3 is selected from substituted or non-substituted heterocyclic group.

18. The device according to claim 17, wherein the organic emitting layer further includes an electron injection layer between the electron transporting-injection layer and the second electrode.
